(19) United States
(12) Patent Application Publication
(10) Pub. No.: US 2009/0117662 A1
(43) Pub. Date: May 7, 2009

(54) Mutants of IGF Binding Proteins and Methods of Production of Antagonists Thereof

(76) Inventors: Hans-Georg Beisel, Moelndal (SE); Dirk Demuth, Muenster-Sarmsheim (DE); Richard Engh, Wessling (DE); Tadeusz Holak, Martinsried (DE); Robert Huber, Germering (DE); Kurt Lang, Penzberg (DE); Ralf Schumacher, Penzberg (DE); Wojciech Zeslawski, Krakow (PL)

Correspondence Address: HOFFMANN-LA ROCHE INC.;PATENT LAW DEPARTMENT, 340 KINGSLAND STREET, NUTLEY, NJ, 07110, US

(21) Appl. No.: 12/185,862

(22) Filed: Aug. 5, 2008

Related U.S. Application Data
(62) Division of application No. 10/479,819, filed on Jul. 6, 2004, now abandoned.

(30) Foreign Application Priority Data
Jun. 7, 2001 (EP) 0112958.2

(51) Int. Cl. G01N 33/68; C07K 19/00

(52) U.S. Cl. 436/86; 530/399

(57) ABSTRACT

The present invention provides a crystal suitable for X-ray diffraction, comprising a complex of insulin-like growth factor I or II (IGF) and a polypeptide consisting of the amino acids 40-92 of IGFBP-5 or a fragment thereof consisting at least of the 9^{th} to 12^{th} cysteine of IGFBP-5 methods for the determination of the atomic coordinates of such a crystal; IGFBP mutants with enhanced binding affinity for IGF-I and/or IGF-II, and methods to identify and optimize small molecules which displace IGFs from their binding proteins.

### PRIORITY TO RELATED APPLICATIONS

This application is a division of application Ser. No. 10/479,819, filed Jul. 6, 2004, which is a 371 of PCT/EP02/06161, filed Jun. 5, 2002, which claims the benefit of European Application No. 0112958.2, filed Jun. 7, 2001. The entire contents of the above-identified applications are hereby incorporated by reference.

The present invention relates to a complex of an IGF binding protein fragment (IGFBP) with IGF, its uses and to novel IGFBP mutants with a higher affinity than natural IGFBPs for IGF as well as to methods for the production of antagonists for IGFBPs which hinder or reverse complex formation between IGFBPs and IGF.

### INTRODUCTION

Insulin-like growth factors I and II (hereafter also referred to as IGFs or IGF) are members of the insulin superfamily of hormones, growth factors and neuropeptides whose biological actions are achieved through binding to cell surface receptors. IGF actions are regulated by IGF binding proteins (IGFBPs) that act as transporters of IGFs, protect them from degradation, limit their binding to receptors, and maintain a “reservoir” of biologically inactive IGF (Martin, J. L., and Baxter, R. C., IGF binding proteins as modulators of IGF actions; in: Rosenfeld, R. G., and Roberts, C. T. (eds.), The IGF system, Molecular Biology, Physiology, and Clinical Applications (1999), Humana Press, Totowa, pp. 227-255; Jones, J. L., and Clemmons, D. R., Endocr. Rev. 12 (1995) 10-21; Khandwala, H. M., et al., Endocr. Rev. 21 (2000) 215-244; Hwa, V., et al., The IGF binding protein superfamily, In: Rosenfeld, R. G., and Roberts, C. T. (eds.), The IGF system, Molecular Biology, Physiology, and Clinical Applications (1999), Humana Press, Totowa, pp. 315-327). The IGF and growth hormone (GH) axis plays a large part in regulating fetal and childhood somatic growth and several decades of basic and clinical research have demonstrated that it also is critical in maintaining neoplastic growth (Khandwala, H. M., et al., Endocr. Rev. 21 (2000) 215-244). High circulating IGF-I concentrations may also be an important determinant of cancer incidence (Hankinson, S. E., et al., Lancet 351 (1998) 1393-1396; Holly, J., Lancet 351 (1998) 1373-1374; Wolk, A., Lancet 356 (2000) 1902-1903). Virtually every level of the IGF system mediating response on the tumor tissues (IGFs, IGFBPs, IGF receptors) can be targeted for therapeutic approaches (Khandwala, H. M., et al., Endocr. Rev. 21 (2000) 215-244; Fanayan, S., et al., J. Biol. Chem. 275 (2000) 39146-39151; Imai, Y., et al., J. Biol. Chem. 275 (2000) 18188-18194). It should also be mentioned here that IGFBP-3 has IGF-independent anti-proliferative and proapoptotic effects (Wetterau, L. A., et al., Mol. Gen. Metab. 68 (1999) 161-181; Butt, A. J., et al., J. Biol. Chem. 275 (2000) 39174-39181).

IGF-I and IGF-II are 67% identical single polypeptide chains of 70 and 67 amino acids, respectively, sharing with insulin about 40% sequence identity and presumed structural homology. The first 29 residues of IGFs are homologous to the B-chain of insulin (B region, 1-29), followed by 12 residues that are analogous to the C-peptide of proinsulin (C region, 30-41), and a 21-residue region that is homologous to the A-chain of insulin (A region, 42-62). The carboxy-terminal octapeptide (D region, 63-70) has no counterpart in insulins and proinsulins (Murray-Rust, J., et al., BioEssays 14 (1992) 325-331; Baxter, R. C., et al., J. Biol. Chem. 267 (1992) 60-65). The IGFs are the only members of the insulin superfamily in which the C region is not removed proteolytically after translation. The 3D structure of insulin has been studied intensively since the first crystal structure determination in the 1960s (Adams, M. J., et al., Nature 224 (1969) 491-492). There are now structures of insulins in several oligomeric states, for insulins crystallized in different solvent conditions, and for many variants from different species and chemical modifications. This is in stark contrast to IGFs (and proinsulins) for which no high definition structure has been available prior to this report. Instead, the tertiary structure of IGF-I has been modeled after porcine insulin (Blundell, T. L., Proc. Natl. Acad. Sci. USA 75 (1978) 180-184). 2D NMR studies of IGF-I have confirmed that the solution structure is consistent with the model (Cooke, R. M., et al., Biochemistry 30 (1991) 5484-5491; Sato, A., et al., Int. J. Pept. Protein Res. 41 (1993) 433-440). However, NMR studies of IGF-I have yielded structures only of low resolution, probably because IGF-I is soluble at the concentrations required for NMR only at pH values below 3 (Cooke, R. M., et al., Biochemistry 30 (1991) 5484-5491; Sato, A., et al., Int. J. Pept. Protein Res. 41 (1993) 433-440). More recently, better defined structures have been obtained for IGF-II (Terasawa, H., et al., EMBO J. 13 (1994) 5590-5597; Torres, A. M., et al., J. Mol. Biol. 248 (1995) 385-401) and for a Glu-3 to Arg variant of IGF-I (long-[Arg3]IGF-I) that additionally possesses a 13-amino acid extension at the N-terminus (Laajoki, L. G., et al., J. Biol. Chem. 275 (2000) 10009-10015).

IGFBPs (insulin-like growth factor binding proteins −1 to −6) are proteins of 216 to 289 residues, with mature IGFBP-5 consisting of 252 residues (Wetterau, L. A., et al., Mol. Gen. Metab. 68 (1999) 161-181). All IGFBPs share a common domain organization. The highest conservation is found in the N-(residues 1 to ca. 100) and C— (from residue 170) terminal cysteine rich regions. Twelve conserved cysteines are found in the N-terminal domain and six in the C-terminal domain. The central, weakly conserved part (L-domain) contains most of the cleavage sites for specific proteases (Chernausek, S. D., et al., J. Biol. Chem. 270 (1995) 11377-11382). Several different fragments of IGFBPs have been described and biochemically characterized so far (Mazerbourg, S., et al., Endocrinology 140 (1999) 4175-4184). Mutagenesis studies suggest that the high affinity IGF binding site is located in the N-terminal domain (Wetterau, L. A., et al., Mol. Gen. Metab. 68 (1999) 161-181; Chernausek, S. D., et al., J. Biol. Chem. 270 (1995) 11377-11382) and that at least IGFBP-3 and IGFBP-2 contain two binding determinants, one in the N- and one at the C-terminal domains (Wetterau, L. A., et al., Mol. Gen. Metab. 68 (1999) 161-181). Recently, a group of IGFBP-related proteins (IGFBP-rPs) which bind IGFs with lower affinity than IGFBPs have been described (Hwa, V., et al., The IGF binding protein superfamily, In: Rosenfeld, R. G., and Roberts, C. T. (eds.), The IGF system, Molecular Biology, Physiology, and Clinical Applications (1999), Humana Press, Totowa, pp. 315-327). IGFBPs and IGFBP-rPs share the highly conserved and cysteine-rich N-terminal domain which appears to be crucial for several biological actions, including their binding to IGFs and high affinity binding to insulin (Hwa et al., 1999). N-terminal fragments of IGFBP-3, generated for example by plasma digestion, also bind insulin and physiologically are thus likely relevant for insulin action. Beyond the N-terminal domain, there is a lack of sequence similarity between the IGFBPs and IGFBP-rPs.

The sequences of human IGFBP-1 to -6 are described in detail in the SwissProt Database (http://www.expasy.ch) and identified by the following Accession Nos.:

| Name | Accession No. |
|---|---|
| IGFBP-1 | P 08833 |
| IGFBP-2 | P 18065 |
| IGFBP-3 | P 17936 |
| IGFBP-4 | P 22692 |
| IGFBP-5 | P 24593 |
| IGFBP-6 | P 24592 |

The amino acid positions described in the following refer to the sequence of the mature forms the human IGF binding proteins (sequence after removal of the signaling peptide starts with amino acid in position 1, see also Tables 1 to 6).

The association of insulin-like growth factors with neoplasia indicates that inhibition of the IGF signaling pathway in tumors might be a successful strategy in cancer therapy. Such modulation might be accomplished, for example, through exogenous administration of recombinant inhibitory IGFBPs and effective fragments thereof. Additionally, tumor cell IGFBP production, inhibition or degradation may be controlled by agents such as tamoxifen and ICI 182,780 that modify tumor IGFBP production (Khandwala, H. M., et al., Endocr. Rev. 21 (2000) 215-244). The consequent alteration in IGFBP-3 levels appears in certain instances to inhibit IGF-1-stimulated cell proliferation (Khwandala et al., 2000). There is also recent evidence that IGFBP-3 may be a p53-independent effector of apoptosis in breast cancer cells via its modulation of the Bax:Bcl-2 protein ratio (Butt, A. J., et al., J. Biol. Chem. 275 (2000) 39174-39181; Wetterau, L. A., et al., Mol. Gen. Metab. 68 (1999) 161-181).

IGFBPs show a significant inhibition of tumor cell proliferation in vitro but only very high doses result in inhibition of tumor growth in vivo (van den Berg, C. L., et al., Eur. J. Cancer 33 (1997) 1108-1113). Van den Berg therefore covalently coupled IGFBP-1 to polyethylene glycol, which leads to a prolonged serum half-life. However, the inhibitory effects of the pegylated IGFBP-1 is still not sufficient for therapeutic intervention in humans because only partial response is observed even if pegylated IGFBP-1 is given in doses of 1 mg/dose daily in mice. This corresponds to a dose of 50 mg/kg×day which can not be administered to humans by established procedures and can not be produced economically.

IGF releasing peptides are described by Loddick, S. A., et al., Proc. Natl. Acad. Sci. USA 95 (1998) 1894-1898 and Lowman, H. B., et al., Biochemistry 37 (1998) 8870-8878. The described molecules which are able to displace IGFs from their binding proteins are either mutants of IGF-I which bind to IGFBPs but are not able to stimulate the IGF-IR or a 14 amino acid peptide with similar properties derived from a phage-display library. The biological activities of the peptides were shown by administration either by injection into the lateral ventricle of the brain or infused by a minipump.

Mutagenesis studies for IGFs indicated that IGF amino acid residues Glu 3, Thr 4, Gln 15 and Phe 16 of IGF-I and Glu 6, Phe 48, Arg 49 and Ser 50 in IGF-II are important for binding to IGFBPs (Baxter, R. C., et al., J. Biol. Chem. 267 (1992) 60-65; Bach, L. A., et al., J. Biol. Chem. 268 (1993) 9246-9254; Luethi, C., et al., Eur. J. Biochem. 205 (1992) 483-490; Jansson, M., et al., Biochemistry 36 (1997) 4108-4117). Baxter et al. (1992) suggested that the IGF-I amino acid residues Glu 3, Thr 4, Gln 15 and Phe 16 are crucial for interaction with IGFBP-3, whereas residues Phe 49, Arg 50 and Ser 51 are of secondary importance. It also was suggested that Phe 26 of IGF-II plays a role in changing the local structures of IGFs but does not directly bind to IGFBPs (Terasawa, H., et al., EMBO J. 13 (1994) 5590-5597).

Kalus, W., et al., in EMBO J. 17 (1998) 6558-6572, describe proteolytic studies of human IGFBP-5 and the cloning and expressing of the domain of IGFBP-5 between residues 40-92 (mini-IGFBP-5); this domain binds IGF-I and IGF-II with K_{D} values of 37 nM and 6 nM, respectively, as well as the determination of the solution structure of uncomplexed mini-IGFBP-5 by NMR. Kalus et al. identified some IGF binding sites which are residues Val49, Tyr50, Pro62 and Lys68 to Leu75 of IGFBP-5.

Imai, Y., et al., in J. Biol. Chem. 275 (2000) 18188-18194, describe an IGFBP-3 variant and an IGFBP-5 variant, each with a five-fold substitution pattern at amino acid positions hypothesized by Kalus et al. as IGF binding sites. Imai et al. found that a substantial alteration of the amino acid residues simultaneously at positions 68, 69, 70, 73 and 74 results in a 1000-fold or larger reduction in the affinity for IGF-I in relation to the affinity of wild-type IGFBP-5.

Conover, C. A., et al., in J. Biol. Chem. 270 (1995) 4395-4400, describe protease-resistant mutants of IGFBP-4. All four IGFBP-4 mutants around the putative cleavage site at Met135-Lys136 and the wild-type protein bind IGFs with equivalent affinities.

Byun, D., et al., in J. Endocrinology 169 (2001) 135-143, postulate several regions involved in IGF binding by IGFBP-4. Deletion of segments Leu72-Ser 91 or Leu72-His74 results in loss of IGF binding. Also mutation of certain cysteine residues significantly reduces the binding of IGFs.

Thus, these described mutant forms of insulin-like growth factor binding proteins have reduced or equivalent affinities for IGF-I and/or IGF-II. Mutants of IGFBPs with a significantly higher affinity and a therefore improved effectiveness have not been known heretofore and there exists a need for such molecules as well as for methods for identifying IGFBP antagonists.

### SUMMARY OF THE INVENTION

The invention provides a crystal suitable for X-ray diffraction, comprising a complex of insulin-like growth factor I or II and a polypeptide consisting of the amino acids 39-91 of IGFBP-1, the amino acids 55-107 of IGFBP-2, the amino acids 47-99 of IGFBP-3, the amino acids 39-91 of IGFBP-4, the amino acids 40-92 of IGFBP-5, or the amino acids 40-92 of IGFBP-6 or a fragment thereof consisting at least of the 9^{th} to 12^{th} cysteine of IGFBP-1, IGFBP-2, IGFBP-3, IGFBP-4, or IGFBP-5 or at least of the 7^{th} to 10^{th} cysteine of IGFBP-6 (such polypeptides and fragments are hereinafter also referred to as “mini-IGFBPs).

Such a crystal is suitable for determining the atomic coordinates of the binding sites of IGF-I, IGF-II, and IGFBPs, and therefore allows the optimization of these molecules to identify and improve stabilizing interactions between IGF-I or IGF-II and IGFBPs. Preferably, the crystal effectively diffracts X-ray for the determination of the atomic coordinates of said complex to a resolution of 1.5 to 3.5 Å. The crystal is arranged in the cubic space group P2₁3 having unit cell dimensions of 74.385 Å×74.385 Å×74.385 Å.

The invention further provides a method for producing a crystal suitable for X-ray diffraction, comprising
(a) contacting IGF-I or IGF-II with a polypeptide consisting of the amino acids 39-91 of IGFBP-1, the amino acids 55-107 of IGFBP-2, the amino acids 47-99 of IGFBP-3, the amino acids 39-91 of IGFBP-4, the amino acids 40-92 of IGFBP-5, or the amino acids 40-92 of IGFBP-6 or a fragment thereof consisting at least of the 9^{th} to 12^{th} cysteine of IGFBP-1, IGFBP-2, IGFBP-3, IGFBP-4, or IGFBP-5 or at least of the 7^{th} to 10^{th} cysteine of IGFBP-6, to form a complex which exhibits restricted conformation mobility, and
(b) obtaining a crystal from the complex so formed suitable for X-ray diffraction.

Using this crystal, the atomic coordinates of the complex can be determined.

The invention further comprises a method for identifying a mutant of IGFBP or a mutant of a fragment thereof consisting at least of the 9^{th} to 12^{th} cysteine of IGFBP-1, IGFBP-2, IGFBP-3, IGFBP-4, or IGFBP-5 or at least of the 7^{th} to 10^{th} cysteine of IGFBP-6, and having enhanced binding affinity for IGF-I and/or IGF-II comprising
(a) constructing a three-dimensional structure of the complex of IGF-I or IGF-II and a polypeptide consisting of the amino acids 39-91 of IGFBP-1, the amino acids 55-107 of IGFBP-2, the amino acids 47-99 of IGFBP-3, the amino acids 39-91 of IGFBP-4, the amino acids 40-92 of IGFBP-5, or the amino acids 40-92 of IGFBP-6 or a fragment thereof consisting at least of the 9^{th} to 12^{th} cysteine of IGFBP-1, IGFBP-2, IGFBP-3, IGFBP-4, or IGFBP-5 or at least of the 7^{th} to 10^{th} cysteine of IGFBP-6, based on the atomic coordinates of a crystal consisting of IGF-I or IGF-II and said polypeptide;
(b) employing said three-dimensional structure and modeling methods to identify said mutant in which an amino acid residue within a distance of 5 Å to a hydrophobic amino acid residue of IGF-I or IGF-II is modified in that the hydrophobic interaction between IGF-I or IGF-II and said mutant of IGFBP is enhanced;
(c) producing said mutant;
(d) assaying said mutant to determine said enhanced binding affinity for IGF.

The invention further comprises a method for identifying a mutant of IGFBP-5 with enhanced binding affinity for IGF-I, said method comprising
(a) constructing a three-dimensional structure of the complex of IGF-1 and IGFBP-5 defined by the atomic coordinates shown in FIGS. 5 and 6;
(b) employing said three-dimensional structure and modeling methods to identify an amino acid residue in IGFBP-5 within a distance of 5 Å or shorter to an amino acid residue of IGF-I, wherein said residue of IGFBP-5 can be modified hydrophobically in that the hydrophobic interaction between IGF and IGFBP-5 is enhanced;
(c) producing said mutant;
(d) assaying said mutant to determine said enhanced binding affinity for IGF.

The amino acid residue(s) in which IGFBP(s) is/are modified is/are preferably selected from the amino acids 39-91 of IGFBP-1, the amino acids 55-107 of IGFBP-2, the amino acids 47-99 of IGFBP-3, the amino acids 39-91 of IGFBP-4, the amino acids 49-92 of IGFBP-5, or the amino acids 40-92 of IGFBP-6.

Especially preferred IGFBP mutants are modified at amino acid residues 49, 70 and/or 73 corresponding to IGFBP-5 sequence alignment and according to Table 7.

The invention therefore provides mutant IGFBPs (“IGFBPs” as used herein means IGFBP-1, IGFBP-2, IGFBP-3, IGFBP-4, IGFBP-5 and/or IGFBP-6) with enhanced affinity (preferably about 3-fold to 10-fold increased affinity to the corresponding wild-type IGFBP) for IGF (“IGF” as used herein means IGF-I and/or IGF-II), improved inhibitory potency for the activity of IGF in vitro and in vivo and therefore improved therapeutic effectiveness.

The invention further provides a method for identifying a compound capable of binding to IGFBP, comprising
(a) constructing a three-dimensional structure of a complex of IGF-I or IGF-II and a polypeptide consisting of the amino acids 39-91 of IGFBP-1, amino acids 55-107 of IGFBP-2, amino acids 47-99 of IGFBP-3, amino acids 39-91 of IGFBP-4, amino acids 40-92 of IGFBP-5, amino acids 40-92 of IGFBP-6 or a fragment thereof consisting at least of the 9^{th} to 12^{th} cysteine of IGFBP-1, IGFBP-2, IGFBP-3, IGFBP-4, or IGFBP-5 or at least of the 7^{th} to 10^{th} cysteine of IGFBP-6, based on the atomic coordinates of a crystal consisting of IGF-I and said IGFBP;
(b) employing said three-dimensional structure and modeling methods to identify a compound forming a complex with said IGFBP by hydrophobic binding with amino acids 49, 50, 70, 71 and 74 in the case of IGFBP-5 and in the case of IGFBP-1, IGFBP-2, IGFBP-3, IGFBP-4 and IGFBP-6 with corresponding amino acids according to Table 7;
(c) producing said compound;
(d) determining the binding between the compound and IGFBP.

The invention further provides a method of inhibiting the binding of IGF to the IGFBP in a subject, preferably a human subject, comprising administering an effective amount of an above-described mutant of IGFBP to the subject.

### DETAILED DESCRIPTION OF THE INVENTION

The present invention provides methods for co-crystallizing IGF-I or IGF-II with a truncated N-terminal fragment of IGFBP, preferably of IGFBP-5 (mini-IGF), where the crystals diffract X-rays with sufficiently high resolution to allow determination of the three-dimensional structure of said complex, including atomic coordinates. The three-dimensional structure (e.g. as provided on computer-readable media) is useful for rational drug design of IGFBP mutants with modified affinity for IGF-I or IGF-II, preferably with an improved affinity. There is specifically provided a method for co-crystallizing IGF-I with a polypeptide consisting of an isolated folded domain of IGFBPs (mini-IGFBPs), which is formed by the amino acids between the 9^{th} and the 12^{th} cysteine of IGFBP-1 to IGFBP-5 or the 7^{th} and 10^{th} cysteine of IGFBP-6 and additionally including up to 7 amino acids N-terminal of this fragment and up to 5-20 amino acids C-terminal to this fragment. The amino acids 39-91 of BP-1, the amino acids 55-107 of IGFBP-2, the amino acids 47-99 of IGFBP-3, the amino acids 39-91 of IGFBP-4, the amino acids 40-92 of IGFBP-5, or the amino acids 40-92 of IGFBP-6 or fragments thereof are especially suitable to form a complex with IGF-I or IGF-II which exhibits restricted conformational mobility and high suitability for X-ray diffraction.

Such a complex co-crystallizes in a manner sufficient for the determination of atomic coordinates by X-ray diffraction. The crystal effectively diffracts X-ray for the determination of the atomic coordinates of the complex to a resolution of 1.5 or at least better (less) than 3.5 Å. Said IGFBP fragments are able to form a compact and globular structure whose scaffold is secured by an inside packing of two cysteine bridges and stabilized further by a three-stranded β-sheet. The folded fragments are still able to bind IGF-I and IGF-II with high affinities. Other forms of the IGFBPs such as full-length IGFBPs, the isolated C-terminal domain of IGFBPs or fragments without N-terminal truncation do not co-crystallize with IGF in a suitable manner for X-ray-based determination of the structure at high resolution.

Knowledge of the crystal structure enables the production of specific IGFBP mutants which develop improved interaction with, thereby exhibiting enhanced affinity for, IGF and, as a consequence, have improved therapeutic efficacy as IGF antagonists. Such IGFBP mutants with increased affinity for IGF are capable of preventing the formation of the complex between naturally occurring IGF and IGF-I receptor (IGF-IR) in vitro and in vivo and, thereby, of effecting an decrease in the concentration of biologically active, free IGF. Such rational designed IGF antagonists are therefore capable of inhibiting tumor growth and inducing apoptosis in tumor cells more efficient than natural IGFBPs. As a result, lower doses of the optimal designed IGFBP mutants with enhanced affinity are needed for achieving an effect comparable to that of naturally occurring IGFBPs.

A further embodiment of the invention is the identification and optimization of non-proteinaceous compounds which bind to the IGF binding site of IGFBPs and prevent the formation of an inhibitory complex between IGFs and IGFBPs and therefore activates the anabolic action of IGF. Such “IGF-releasing compounds” can be identified according to the invention on the basis of the crystal data, using protein-ligand docking programs such as FlexX (Kramer, B., et al., Proteins: Structure, Functions and Genetics 37 (1999) 228-241).

The X-ray diffraction patterns of the invention have a sufficiently high resolution to be useful for three-dimensional modeling of an IGF releasing compound. Preferably, the resolution is in the range of 1.5 to 3.5 Å, preferably 1.5 to 3.0 Å. Three-dimensional modeling is performed using the diffraction coordinates from these X-ray diffraction patterns. The coordinates are entered into one or more computer programs for molecular modeling as known in the art. Such molecular modeling can utilize known X-ray diffraction molecular modeling algorithms or molecular modeling software to generate atomic coordinates corresponding to the three-dimensional structure of at least one IGF releasing compound.

Such a compound shows affinity for IGFBP based on stereochemical complementary relative to naturally occurring IGFs. Such stereochemical complementary according to the present invention is characterized by a molecule that matches intra-site surface residues that form the contours of IGFBPs as enumerated by the coordinates set out in FIGS. 5 and 6. The residues that define the contours are depicted in FIGS. 5 and 6. Matching according to the invention means that the identified atoms or atom groups interact with the IGFBP surface residues, for example via hydrogen bonding or by enthalpy-reduced van der Waals interactions which prevent or reduce the interaction between IGFBP and IGFs and thereby promote the release of the biologically active compound from the binding site. In general, the design of a molecule possessing stereochemical complementary to the contours of IGFBPs can be accomplished by means of techniques that optimize either chemically or geometrically the fit between a molecule and a target receptor. Known techniques of this sort are reviewed by Sheridan, R. P., and Venktaraghavan, R., Acc. Chem. Res. 20 (1987) 322; Goodford, P. J., J. Med. Chem. 27 (1984) 557; Verlinde, C., and Hol, W., Structure 2 (1994) 577; and Blundell, T. L. et al., Nature 326 (1987) 347. The design of optimized IGFBP ligands based on the invention is preferably done by the use of software such as GRID (Goodford, P. J., J. Med. Chem. 28 (1985) 849-857), a program that determines probable interaction sites between probes with various functional group characteristics and the protein surface—is used to analyze the surface sites to determine structures of similar inhibiting proteins or compounds.

The program DOCK (Kuntz, I. D., et al., J. Mol. Biol. 161 (1982) 269-288) can also be used to analyze an active site or ligand binding site and suggest ligands with complementary steric properties. Several methodologies for searching three-dimensional databases to test pharmacophore hypotheses and select compounds for screening are available. These include the program CAVEAT (Bacon et al., J. Mol. Biol. 225 (1992) 849-858) which uses databases of cyclic compounds which can act as spacers to connect any number of chemical fragments already positioned in the active site. The program LUDI (Bohm, H. J., et al., J. Comput. Aided Mol. Des. 6 (1992) 61-78 and 593-606) defines interaction sites into which both hydrogen bonding and hydrophobic fragments fit.

Programs suitable for searching three-dimensional databases to identify also non-proteinaceous molecules bearing a desired pharmacophore include: MACCS-3D and ISIS/3D (Molecular Design Ltd., San Leandro, Calif.), ChemDBS-3D (Chemical Design Ltd., Oxford, U.K.), and Sybyl/3DB Unity (Tripos Associates, St. Louis, Mo.).

Programs suitable for pharmacophore selection and design include: DISCO (Abbott Laboratories, Abbott Park, Ill.), Catalyst (Bio-CAD Corp., Mountain View, Calif.), and ChemDBS-3D (Chemical Design Ltd., Oxford, U.K.).

Databases of chemical structures are available from a number of sources including Cambridge Crystallographic Data Centre (Cambridge, U.K.) and Chemical Abstracts Service (Columbus, Ohio).

De novo design programs include Ludi (Biosyrn Technologies Inc., San Diego, Calif.), Sybyl (Tripos Associates) and Aladdin (Daylight Chemical Information Systems, Irvine, Calif.).

Those skilled in the art will recognize that the design of such compounds may require slight structural alteration or adjustment of a chemical structure designed or identified using the methods of the invention.

Non-proteinaceous compounds and IGFBP mutants with increased binding affinity for IGF can be identified by incubating said compounds or mutants with an IGF-I/IGFBP-5 complex and measuring the binding of released IGF-I to IGF-I receptor expressing cells. Due to the binding of IGF-I to its cell-bound receptor, the receptor is activated and autophosphorylated. Alternatively, radiolabeled IGF-I can be used and its binding to its receptor after release from the complex can be determined.

Formation of the IGF-1 mini-IGFBP-5 complex buries a binding surface totaling about 550 Å². Of the eleven IGFBP-5 amino acid residues within 5 Å of IGF, six are hydrophobic, three of which are surface-exposed leucines and valines and are of primary importance for hydrophobic interaction to IGFs (FIGS. 1 to 4). On the IGF side, four of the eleven amino acid residues within 5 Å of mini-IBFBP-5 are hydrophobic (FIGS. 1 to 4).

The IGFBPs bind to IGF-I and IGF-II by presenting a binding surface complementary to that of IGF. The IGF binding surface consists of a relatively flat hydrophobic surface, a small hydrophobic depression, two hydrophobic protruberances, and surrounding polar residues. Identification of the IGF binding surface itself (FIG. 3) enables the design of binding partners in general, and optimization of known binding partners in particular. General binding partners will have at least two of the following features 1 to 4:
1. Non-polar atoms lying approximately in a plane defined by atoms Leu74 CD1 and CD2, Val49 CG1 and CG2, Leu70 CB, and Tyr 50 CB, within a perimeter defined by IGF residues Glu9, Glu3, Leu54, Phe 16 and by BP5 atom Tyr 50 OH and depicted in FIG. 3 such that they present an approximately planar and hydrophobic molecular surface of at least 20 square Angstroms.
2. A non-polar atom or atoms near the positions of Leu 70 CG, CD1 relative to IGF, filling the depression of IGF as seen in the complex structure.
3. Hydrophobic and/or aromatic interactions with the side chains of Phe16, Val17, and/or Leu54 of IGF as defined by a net buried surface area in the complex of at least 20 square Angstroms.
4. Polar (hydrogen bonding and/or charge complementary) interactions, either directly or via bridging solvent molecules, with one or more of the following IGF atoms: Asp12 OD1,2; Glu9 OE1,2; Glu3 OE1,2; Glu58OE1,2; Thr4 O,OG1; Cys52O; Ser51 OG; Asp53OD1,2; Arg55NH1,2,NE; Arg21NH1,2,NE; Val17O; Cys18O; Asp20OD1,2,N; Gln15O,OD1,ND2.

Abbreviations: Letters corresponding to standard amino acid atom naming (according to the International Union of Physicists and Chemists—IUPAC—naming).

### CG: Carbon Cγ

### CB: Carbon Cβ

### OE: Oxygen Oε

### OH: Oxygen Oη

### OD: Oxygen Oδ

### O: Backbone Oxygen

### NH: Nitrogen Nη

### NE: Nitrogen Nε

### N: Backbone Nitrogen

### ND: Nitrogen Nδ

The principal IGF/IGFBP interaction, shown in the example of IGF-1 mini-IGFBP-5 interaction, is a hydrophobic sandwich that consists of interlaced protruding side chains of IGF-I and solvent exposed hydrophobic side chains of the mini-IGFBP-5 (FIGS. 1 to 4). The side-chains of IGF-I Phe 16, Leu 54 and also Glu 3, are inserted deep into a cleft on the mini-IGFBP-5 (FIGS. 1 to 4). This cleft is formed by side chains of Arg 53, Arg 59 on the solvent exposed side of the molecule and by Val 49, Leu 70, Leu 74 on the opposite inner side, with a base formed by residues Cys 60 and Leu 61. Phe 16 makes direct contacts with the backbone and side chain of Val 49, and with Cys 60 of mini-IGFBP-5. The hydrophobic cluster is closed on the solvent side by side chains of Glu 3 and Glu 9 of IGF-I and His 71 and Tyr 50 of mini-IGFBP-5. These residues form a network of hydrogen bonds; in addition Arg 59 of mini-IGFBP-5 makes hydrogen bonds with Glu 58 (FIGS. 2 to 4).

Arg 53 and Arg 59 of mini-IGFBP-5 isolate the hydrophobic sandwich from the solvent close to the C-terminus. In the full length IGFBP-5, the segment corresponding to the C-terminus of mini-IGFBP-5 is followed by nine hydrophilic residues and then by at least 30 residues of mixed types. Thus, the conformations seen in the structure of the complex near the C-terminus of mini-IGFBP-5 are likely to be preserved in the complex of IGF-I with the full length-IGFBP-5. The mini-IGFBP-5 domain begins preferably at residue 40 of full length IGFBP-5.

The hydrophobic residues Val 49, Leu 70 and Leu 73 of IGFBP-5 are crucial for binding to IGFs. Since these residues are highly conserved among all IGFBPs, these hydrophobic interactions dominate the IGF binding properties of all IGFBPs.

The increased inhibitory potency of the mutant IGFBPs and fragments thereof results in the inhibition of the binding to and autophosphorylation of the IGF-IR (as described by Kalus, W., et al., in EMBO J. 17 (1998) 6558-6572) at significantly lower concentrations than observed for the wildtype proteins and the corresponding fragments. The higher potency of the mutant IGFBPs furthermore can be shown by the inhibition of the growth of tumor cells in vitro and in vivo. The growth of several tumor cell lines is known to be significantly reduced by IGFBPs. IGFBP-1 for example inhibits the growth of MCF-7 and MDA-MB-435A cells in vitro and the growth of tumors formed MDA-MB-231 cells in vivo in mice (van den Berg, C. L., et al., Eur. J. Cancer 33 (1997) 1108-1113). IGFBP mutants with increased affinity inhibit the growth of these tumor cells at lower concentrations than the wild type proteins.

The following mutations of IGFBPs are preferred for enhancing binding affinity to IGF (numbering according to IGF-BP5 as aligned in FIG. 1) (standard one-letter abbreviation for amino acids used):

TABLE 1
IGFBP-1

| Amino acid No. | Original amino acid | Preferred mutations¹⁾ |
|---|---|---|
| 48 | V | L, I, M, F, Y, W |
| 49 | A | Y, R, K |
| 52 | R | W, Y, M, F, H |
| 60 | R | Y, W, F |
| 69 | L | Y, W, M, I, F |
| 72 | L | I, Y, W, M, F |
| 73 | T | V, L, Y, W, M, I, F |
| 74 | R | H, D |
| 82 | E | R, K, H, N, Q, S, T, A, G |

TABLE 2
IGFBP-2

| Amino acid No. | Original amino acid | Preferred mutations¹⁾ |
|---|---|---|
| 64 | V | L, I, M, F, Y, W |
| 65 | Y | R, K |
| 68 | R | W, Y, M, F, H |
| 76 | Y | W, F |
| 85 | L | Y, W, M, I, F |
| 86 | Q | T, S, R, K, N, H, Y, C |
| 88 | L | I, Y, W, M, F |
| 89 | V | L, I, Y, W, M, F |
| 90 | M | H, D |

TABLE 3
IGFBP-3

| Amino acid No. | Original amino acid | Preferred mutations¹⁾ |
|---|---|---|
| 56 | I | L, V, M, F, Y, W |
| 57 | Y | R, K |
| 60 | R | W, Y, M, F, H |
| 68 | Q | L, Y, W, F |
| 75 | R | Q |
| 77 | L | Y, W, M, I, F |
| 78 | Q | T, S, R, K, N, H, Y, C |
| 80 | L | I, Y, W, M, F |
| 81 | L | Y, W, M, I, F |

TABLE 4
IGFBP-4

| Amino acid No. | Original amino acid | Preferred mutations¹⁾ |
|---|---|---|
| 48 | V | L, I, M, F, Y, W |
| 49 | Y | R, K |
| 52 | R | W, Y, M, F, H |
| 60 | Y | W, F |
| 67 | K | Q |
| 69 | L | Y, W, M, I, F |
| 72 | L | I, Y, W, M, F |
| 73 | M | Y, W, I, F |
| 74 | H | D |

TABLE 5
IGFBP-5

| Amino acid No. | Original amino acid | Preferred mutations¹⁾ |
|---|---|---|
| 49 | V | L, I, M, F, Y, W |
| 50 | Y | R, K |
| 53 | R | W, Y, M, F, H |
| 61 | L | Y, W, F |
| 68 | K | Q |
| 70 | L | Y, W, M, I, F |
| 73 | L | I, Y, W, M, F |
| 74 | L | Y, W, M, I, F |
| 75 | H | D |
| 83 | E | R, K, H, N, Q, S, T, A, G |

TABLE 6
IGFBP-6

| Amino acid No. | Original amino acid | Preferred mutations¹⁾ |
|---|---|---|
| 49 | V | L, I, M, F, Y, W |
| 50 | Y | R, K |
| 53 | N | R, W, Y, M, F, H |
| 61 | H | L, Y, W, F |
| 68 | A | K, Q |
| 70 | L | Y, W, M, I, F |
| 71 | R | T, S, H, K, N, Q, Y, C |
| 73 | L | I, Y, W, M, I, F |
| 74 | L | Y, W, M, I, F |
| 75 | L | H, D |

¹⁾Amino acids are given in the standard one-letter amino acid code and are to be understood as alternative amino acid exchanges (or). For instance, the last line of Table 6 means that amino acid residue 75 of IGFBP-6, which is leucine (L), can preferably be modified to be either histidine (H) or aspartic acid (D). Table 6 is additionally to be interpreted such that amino acids 49, 50, 53, 61, 68, 70, 73, 74 and/or 75 can be exchanged in order to improve affinity. Especially preferred are IGFBP mutants with single point mutations. Most preferred are IGFBP mutants having a single point mutation from the bold face residues. This applies correspondingly to the other tables.

TABLE 7
Sequence alignment showing corresponding amino acids of IGFBP-1 to -6
Amino Acid No.

| IGFBP-1 | IGFBP-2 | IGFBP-3 | IGFBP-4 | IGFBP-5 | IGFBP-6 |
|---|---|---|---|---|---|
| 48 | 64 | 56 | 48 | 49 | 49 |
| 49 | 65 | 57 | 49 | 50 | 50 |
| 52 | 68 | 60 | 52 | 53 | 53 |
| 60 | 76 | 68 | 60 | 61 | 61 |
| 67 | 83 | 75 | 67 | 68 | 68 |
| 69 | 85 | 77 | 69 | 70 | 70 |
| 70 | 86 | 78 | 70 | 71 | 71 |
| 72 | 88 | 80 | 72 | 73 | 73 |
| 73 | 89 | 81 | 73 | 74 | 74 |
| 74 | 90 | 82 | 74 | 75 | 75 |

The presented structure enables in silico screens for small IGFBP ligand inhibitors with the potential to release “free” bioactive IGF. Displacement of IGF from their binding proteins are therapeutically useful in treating a variety of potential indications, including short stature, renal failure, type I and type II diabetes, stroke and other neuro-degenerative diseases.

The compounds and IGFBP mutants of the present invention can be formulated according to methods for the preparation of compositions, preferably pharmaceutical compositions, which methods are known to the person skilled in the art. Preferably, such a compound and IGFBP mutant is combined in a mixture with a pharmaceutically acceptable carrier. Such acceptable carriers are described in, for example, Remington's Pharmaceutical Sciences, 18^{th} ed., 1990, Mack Publishing Company, edited by Oslo et al. (e.g. pp. 1435-1712). Typical compositions contain an effective amount of a non-proteinaceous compound or IGFBP mutant according to the invention, for example from about 1 to 10 mg/ml, together with a suitable amount of a carrier. The compounds and IGFBP mutants may be administered preferably parenterally.

The invention further provides pharmaceutical compositions containing a non-proteinaceous compound or IGFBP mutant according to the invention. Such pharmaceutical compositions contain an effective amount of a compound and IGFBP mutant of the invention, together with pharmaceutically acceptable diluents, preservatives, solubilizers, emulsifiers, adjuvants and/or carriers. Such compositions include diluents of various buffer contents (e.g., acetate, phosphate, phosphate-buffered saline), pH and ionic strength, additives such as detergents and solubilizing agents (e.g., Tween®80, polysorbate, Pluronic®F68), antioxidants (e.g., ascorbic acid, sodium metabisulfite), preservatives (Timersol®, benzyl alcohol) and bulking substances (e.g., saccharose, mannitol).

Compositions and pharmaceutical compositions according to the invention are manufactured in that the substances in pure lyophilized form are dissolved at a concentration up to from 1 to 20 mg/l in PBS or physiological sodium chloride solution at a neutral pH value. For better solubility it is preferred to add a detergent.

Typically, in a standard cancer treatment regimen, patients are treated with dosages in the range of between 0.5 to 10 mg substance/kg weight per day.

The following examples, references, sequence listing and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1A: Sequence alignment of IGF-I (SEQ ID NO: 9) and IGF-II (SEQ ID NO: 10). Bold underlined residues of IGF-I make contacts with mini-IGFPB5. Residues responsible for binding to the IGF-I receptor (IGF-IR) are marked with an asterisk above the sequence.

FIG. 1B: Multiple sequence alignment of the N-terminal domains of human IGF-BPs 1-6 (SEQ ID NOS 11-16, respectively, in order or appearance). The mini-BP construct, numbered according to BP5 numbering, is marked above the aligned residues with “m”, including GS which indicate additional residues from the cloning vector. (After position 81, mini-BP5 was disordered in the X-ray structure; this is indicated with italics.) BP5 residues that interact with IGF-I are shown underlined and in bold face. The degree of conservation of the residues is marked under the alignment with * for strict conservation, : for strict conservation of residue type, and . for relatively high conservation. The consensus sequence uses the following code to depict level of strict conservation: o alcohol, l aliphatic, a aromatic, c charged, h hydrophobic, −negative, p polar, +positive, s small, u tiny, t turnlike).

FIG. 2: The overall structure of the IGF-I (tube model) mini-IGFBP5 (molecular surface) complex. Side chains plotted show the IGF residues in contact with BP5. Particularly important is Phe16, seen filling a hydrophobic depression on the BP5 surface.

FIG. 3: Similar to FIG. 2, whereby the IGF is depicted with its molecular surface and BP5 is depicted as a tube model. Side chains of BP5 responsible for binding to IGF are also depicted. The surface of IGF Phe16 is prominent, as is the relatively flat hydrophobic IGF surface contributing to the interface.

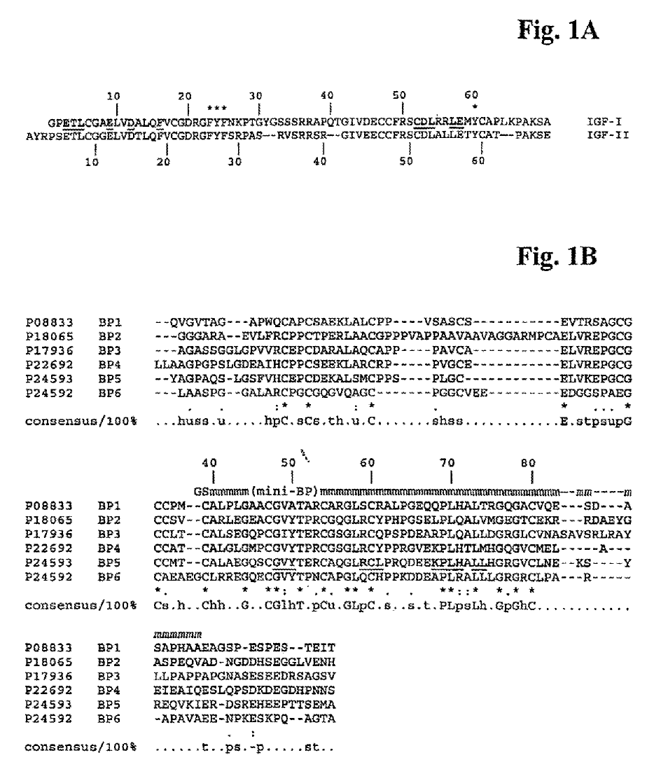

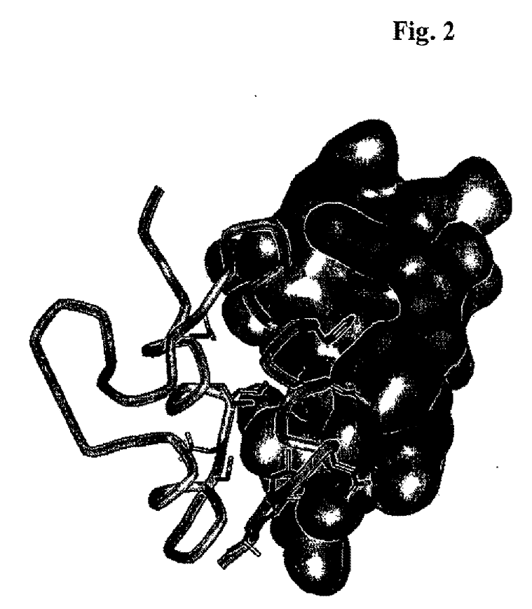

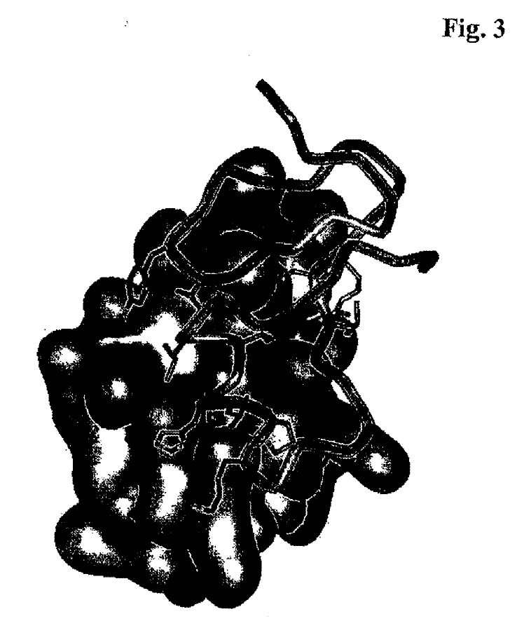

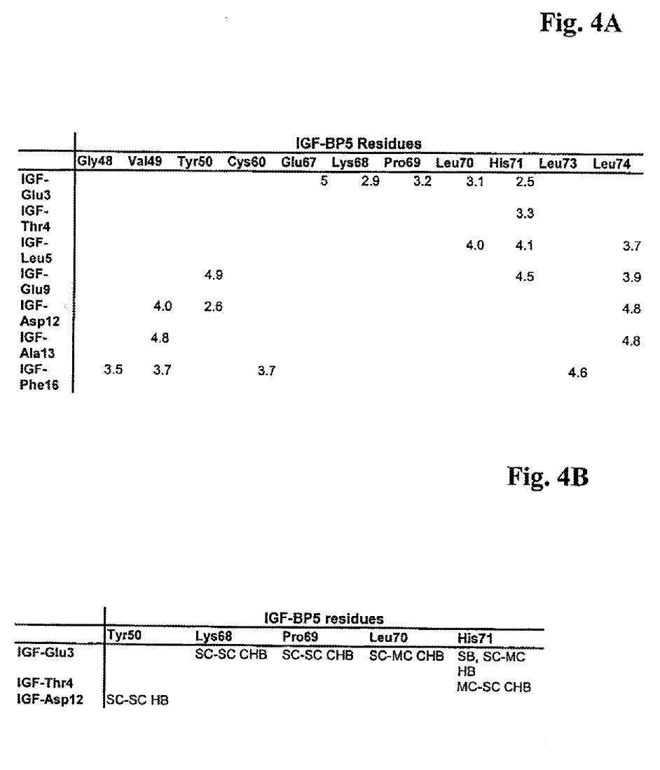

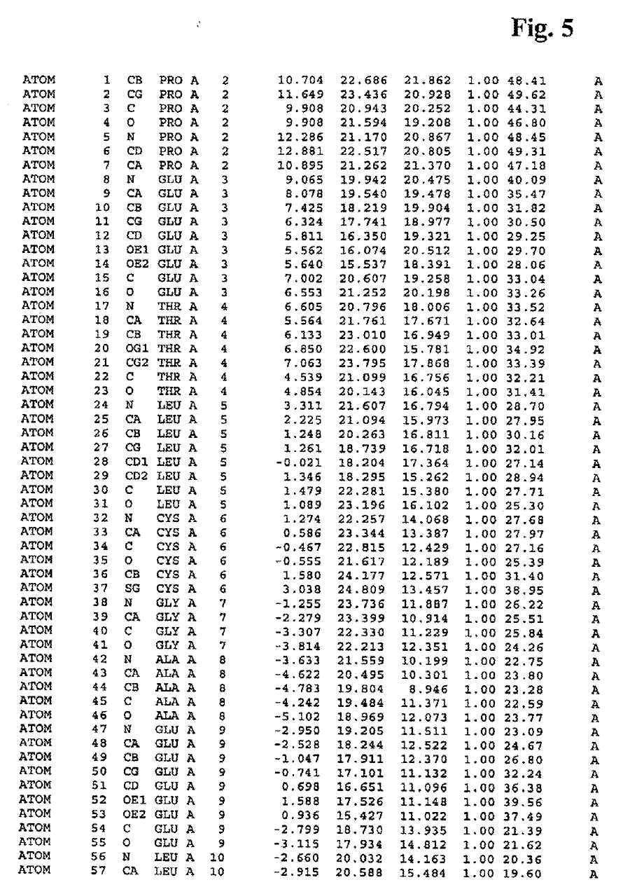

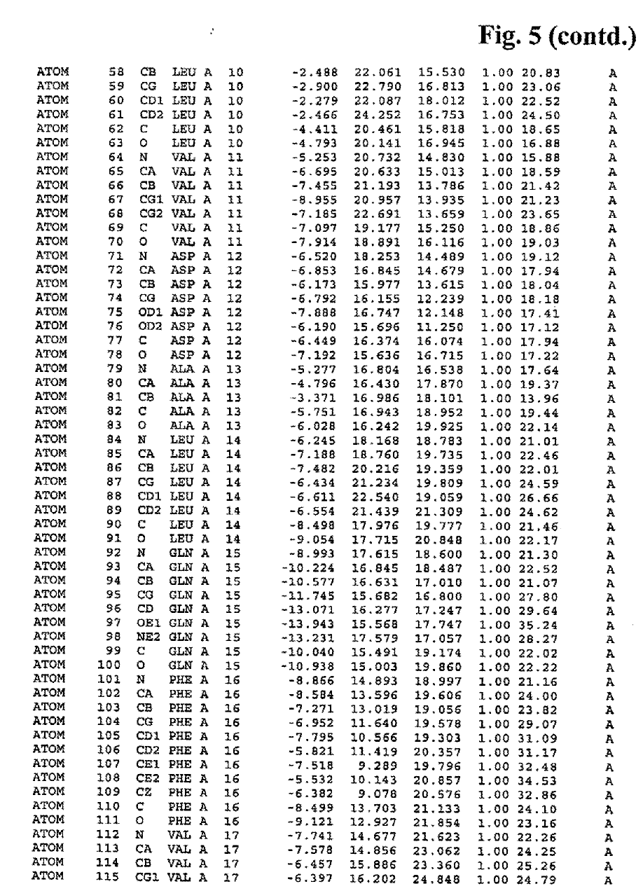

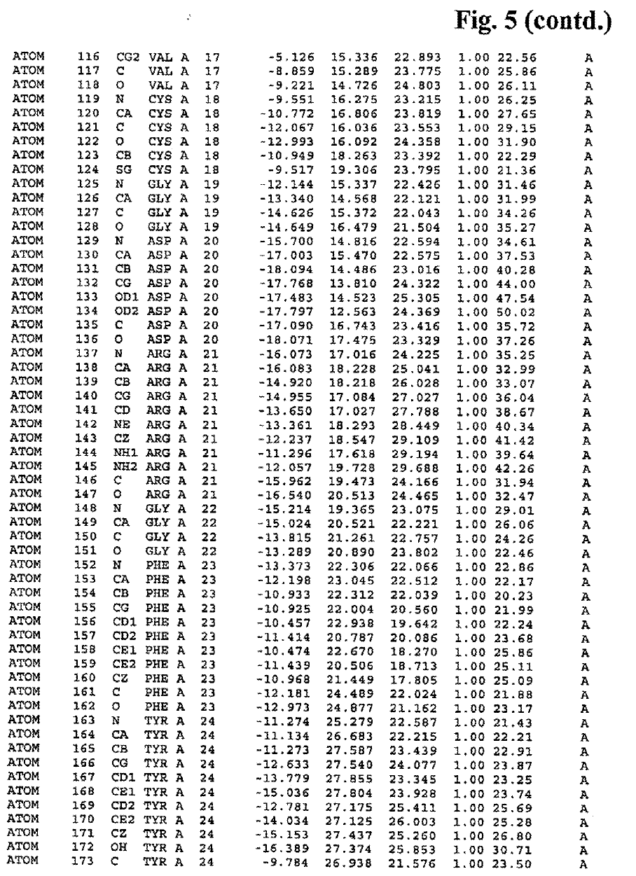

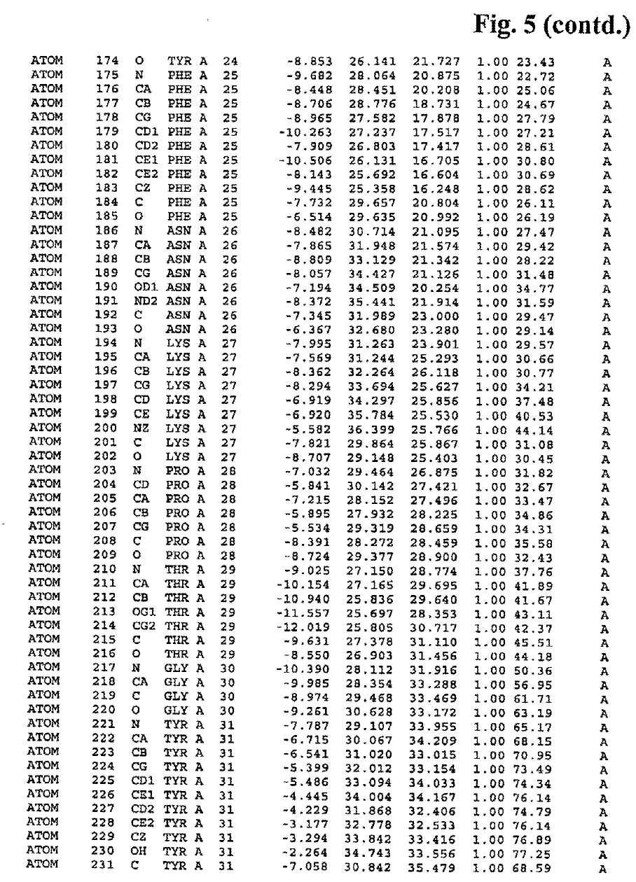

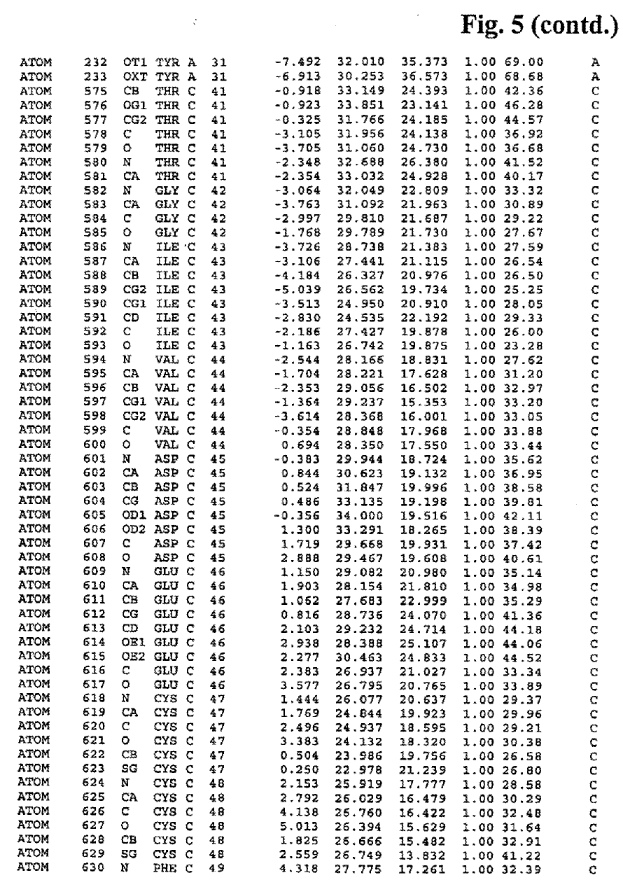

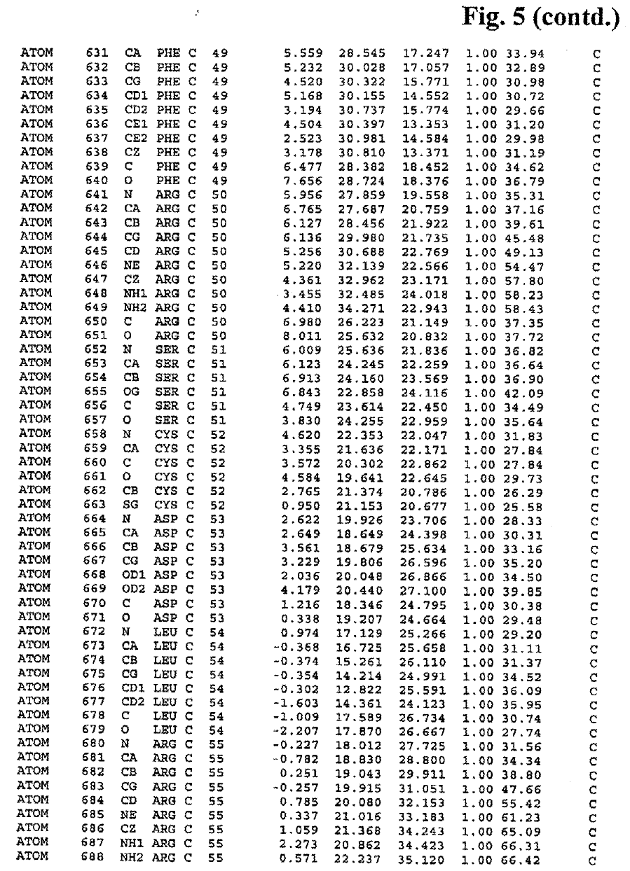

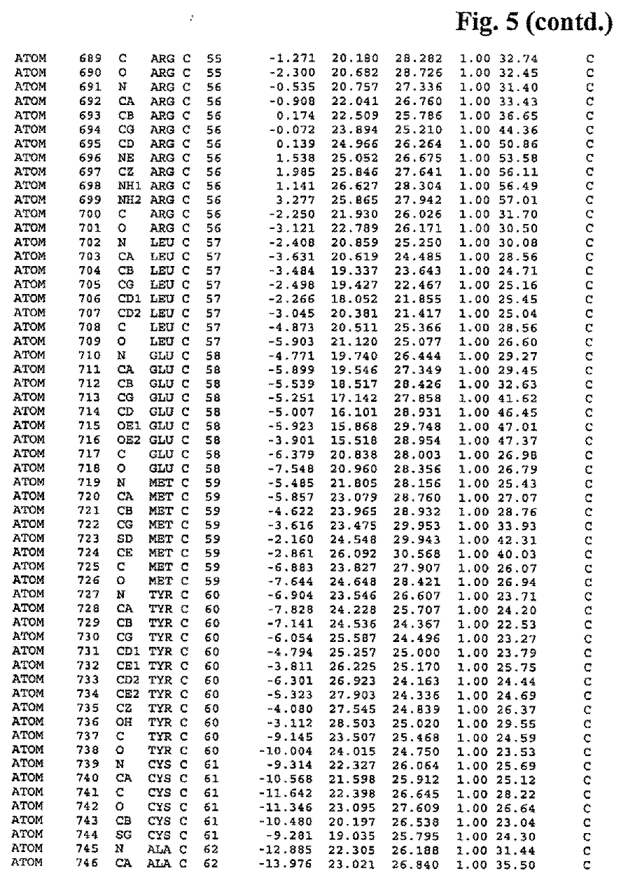

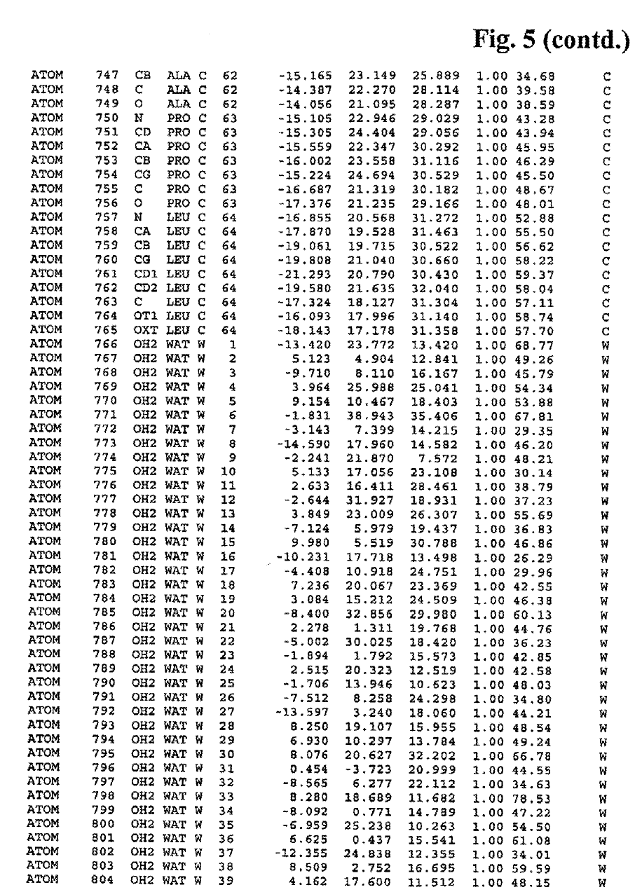

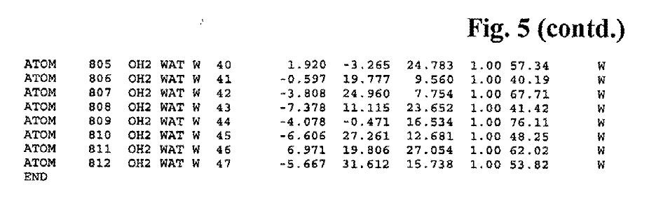

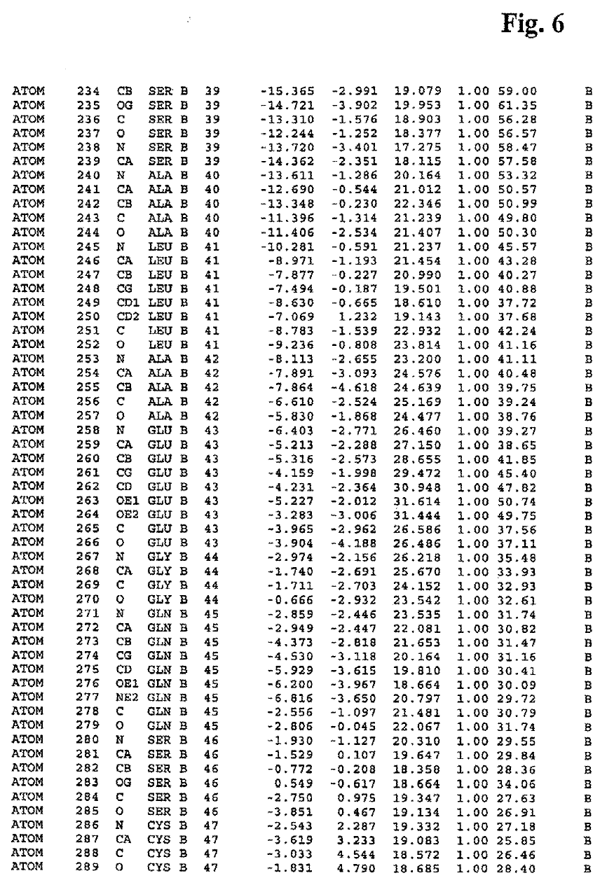

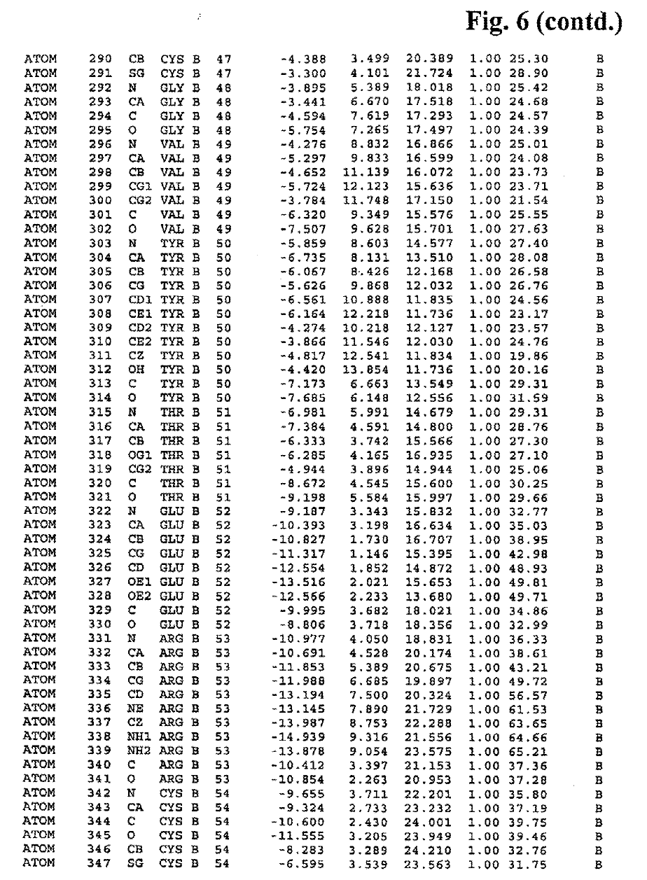

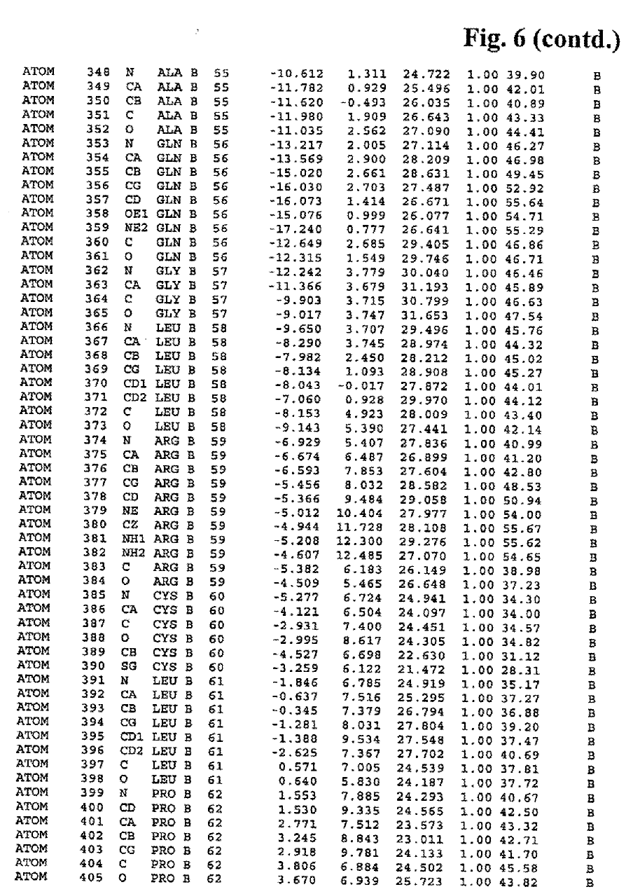

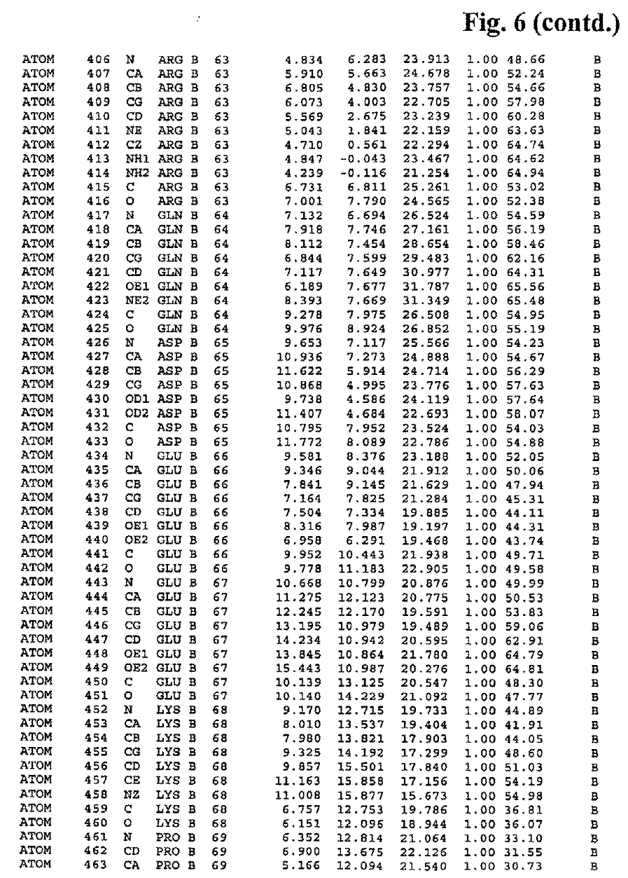

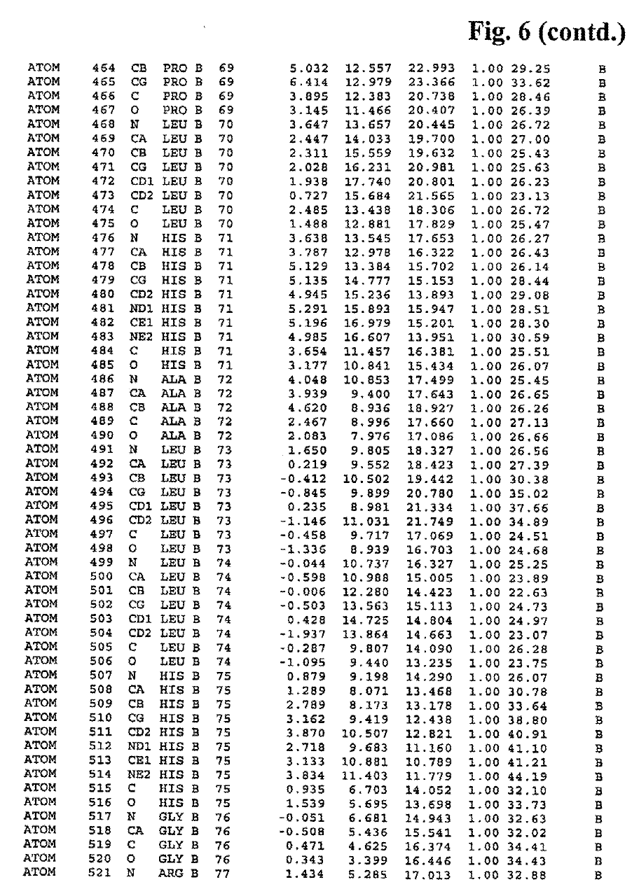

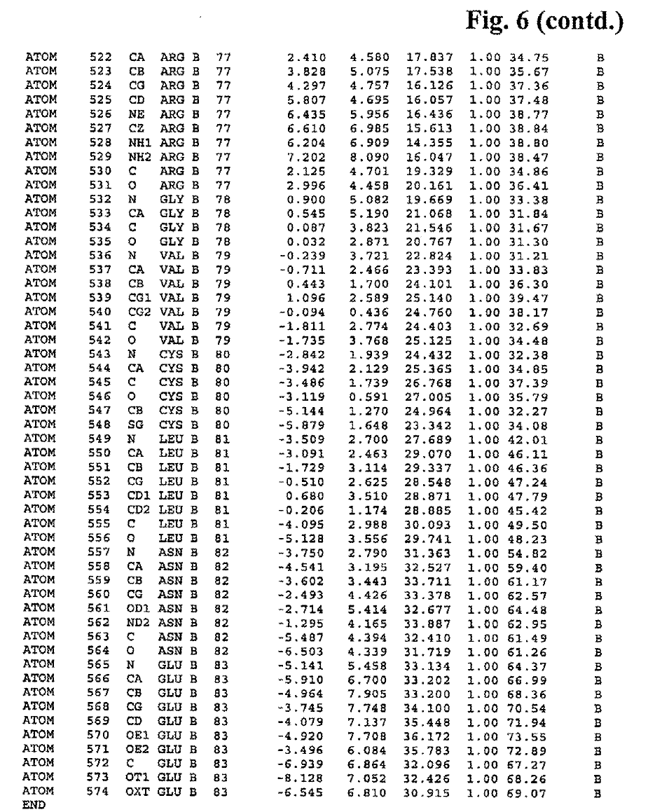

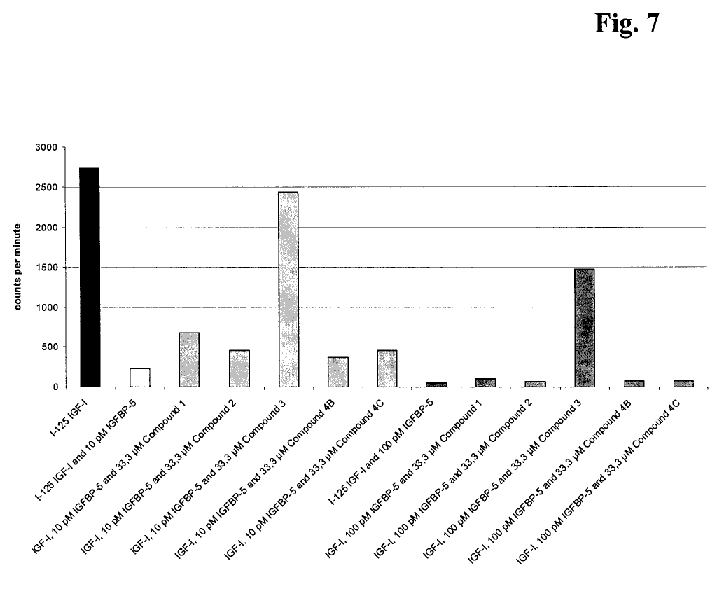

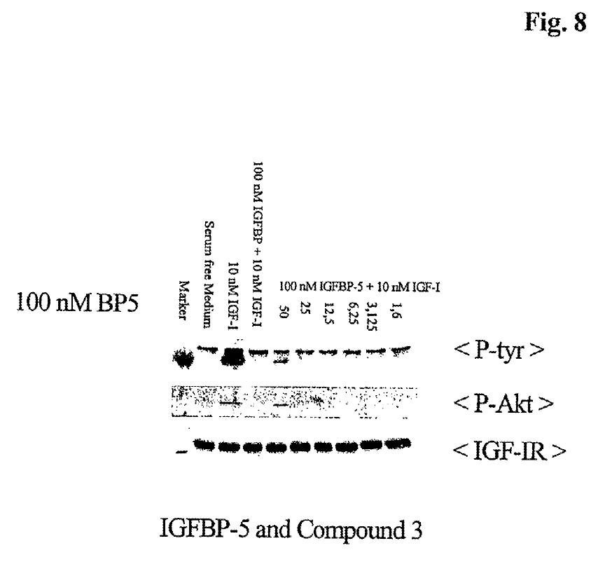

FIGS. 4A and 4B: Summary of IGF-BP5 and IGF-I contacts. Interactions contributing to the binding affinity consist of hydrophobic interactions (a) (involving especially residues Leucines 70, 73, and 74 of BP5 and Phe16 of IGF-I) and also polar interactions (b). Enhancement of BP-IGF binding relies especially on the enhancement of hydrophobic interactions, either by increasing the intermolecular contact surface with these or with additional residues, or by the introduction of further polar contacts.
(A) Packing contacts between IGFBP-5 and IGF-I. Contacts are denoted according to nearest distances, whereby the closest contacts include polar interactions.
(B) Polar contacts between IGFBP-5 and IGF-I. Abbreviations denote hydrogen bonds (HB), CH—O hydrogen bonds (CHB), salt bridge (SB), and side chain (SC) or main chain (MC) interactions.

FIG. 5: Atomic coordinates of IGF-I in the complex with mini-IGFBP-5.

FIG. 6: Atomic coordinates of mini-IGFBP-5 in the complex with IGF-I.

FIG. 7: Binding of radioactive J-125 IGF-I to NIH 3T3 cells expressing the IGF-IR in the absence and in the presence of IGFBP-5 and compounds potentially interfering with complex formation between IGF-I and IGFBP-5

FIG. 8: IGF-I induced autophosphorylation of the IGF-IR expressed by NIH 3T3 cells in the absence and in the presence of IGFBP-5 and compounds potentially interfering with complex formation between IGF-I and IGFBP-5

### Sequence Listing

### SEQ ID NO:1 Primer FBP5LY.

### SEQ ID NO:2 Primer RBP5LY.

### SEQ ID NO:3 Primer FBP5LM.

### SEQ ID NO:4 Primer RBP5LM.

### SEQ ID NO:5 Primer IBP4NdeI.

### SEQ ID NO:6 Primer IBP4BamHI.

### SEQ ID NO:7 Peptide GSALA.

### SEQ ID NO:8 Peptide GSHMDEAIH.

### EXAMPLE 1

### Crystallization, Data Collection and Derivatization

Mini-IGFBP-5 was produced as described by Kalus, W., et al., in EMBO J. 17 (1998) 6558-6572 and in Example 6, and IGF-I was obtained from OvoPepi, Australia. Crystallization was successful with 10% Jeffamine M-600, 0.1 M sodium citrate, 0.01 M ferric chloride, pH 5.6. Within 11 days, crystals appeared at 4° C., growing to a final size of about 0.3×0.2×0.2 mm3. They belong to the cubic space group P213 and have unit cell dimensions a, b, c=74.385 Å, with one complex molecule per asymmetric unit. Soaking in precipitation buffer with heavy atom compounds yielded a derivative K2PtCl4 (2.7 mM, 3 d) which was interpretable. All diffraction data were collected using a 300 mm MAR Research (Hamburg, Germany) image plate detector mounted on a Rigaku (Tokyo, Japan) RU300 rotating anode X-ray generator with graphite monochromatized CuKα radiation. All image plate data were processed with MOSFLM (Leslie, A. G. W., Molecular Data in Processing, in: Moras, D., Podjarny, A. D., and Thierry, J. C. (eds.), Crystallographic Computing 5 (1991), Oxford University Press, Oxford, UK, pp. 50-61) and the CCP4 program suite (Collaborative Computational Project, Number 4 1994).

### EXAMPLE 2

### Phase Calculation, Model Building and Refinement

The structure of the IGF/mini-IGFBP-5 complex was solved by the single isomorphous replacement (s.i.r.) method using one heavy atom derivative described above. Derivative data was analyzed with the native data set, first using isomorphous difference Patterson maps and employing the Patterson vector superposition methods implemented in SHELX-97 (Sheldrick, G., Tutorial on automated Patterson interpretation to find heavy atoms, in: Moras, D., Podjarny, A. D., and Thierry, J. C. (eds.), Crystallographic Computing 5 (1991), Oxford University Press, Oxford, UK, pp. 145-157). The 2 heavy sites locations were confirmed by difference Fourier methods with appropriate initial single site s.i.r. phases using CCP4 programs. The refinement of heavy atom parameters and calculation of s.i.r. phases were done with SHARP (de la Fortelle, E., and de Bricogne, G., Methods Enzymol. 276 (1997) 472-494). The final parameters are given in Table 8. The initial s.i.r. phases were improved with SOLOMON (Abrahams, J. P., and Leslie, A. G. W., Acta. Cryst. D52 (1996) 30-42) using an solvent fraction of 45%, resulting in a 2.1 Å electron density map that was interpretable. Refinement was performed by conjugate gradient and simulated annealing protocols as implemented in CNS 1.0 (Brünger, A. T., et al., Acta Crystallogr. D54 (1998) 905-921. All protocols included refinement of individual isotropic B-factors and using the amplitude based maximum likelihood target function. The R-factor dropped to 21.8% (Rfree=26.2%, resolution range 16.2-2.1 Å) for the final model including 47 water molecules. The water model was calculated using ARP and verified by visual inspection. The final refinement statistics are shown in Table 8.

TABLE 8
Statistics from the crystallographic analysis

| | Native | K₂PtCl₄ |
|---|---|---|
| Resolution (Å) | 16.2-2.1 | 18.6-2.5 |
| Measurements | 45345 | 32833 |
| Unique measurements | 8035 | 4925 |
| % Complete (last shell/Å) | 99.3 (96.9/2.23-2.11) | 99.9 (95.4/2.64-2.5) |
| R_{sym} (%) (last shell) | 8.2 (44.8) | 8.8 (49.5) |
| R_{Cullis-iso} | — | 0.77 |
| Pᵢₛₒ | — | 1.48 |
| Res. for phase calc. (Å) | — | 18.6-2.5 |
| Mean FOM | — | 0.41 |

Refinement statistics:

| | |
|---|---|
| Resolution range (Å) | 16.2-2.1 |
| reflections in working set | 7522 |
| reflections in test set | 501 |
| R_{cryst} (%) | 21.8 |
| R_{free} (%) | 26.2 |
| Protein atoms (non-H) | 765 |
| Solvent atoms (non-H) | 47 |
| Average B-factor (Å²) | 38.1 |
| r.m.s. ΔB (2Å cutoff) | 3.4 |
| Deviations from ideality | |
| (r.m.s.): | |
| Bond lengths (Å) | 0.013 |
| Bond angles (°) | 1.7 |

$${R}_{sym} = \frac{\sum \vert {I ( h )}_{i} - 〈 I ( h ) 〉 \vert}{\sum 〈 I ( h ) 〉}$$

R_{Cullis-iso}=r.m.s. lack of closure/r.m.s isomorphous difference
Pᵢₛₒ (Phasing power)= [symbol] F_{H} [symbol] /r.m.s. lack of closure for all reflections
Mean FOM=mean figure of merit
R_{cryst}=Crystallographic R-factor for reflections used in refinement
R_{free}=Crystallographic R-factor for reflections not used in refinement
r.m.s.=Root mean square

### EXAMPLE 3

### Determination of the Binding Affinity of IGFBP Mutants

The IGF-binding properties of wildtype and mutant fragments and full-length IGFBPs were quantitatively analyzed by BIAcore biosensor measurements. BIAcore 2000, Sensor Chip SA and HBS were obtained from BIAcore AB (Uppsala, Sweden). All experiments were performed at 25° C. and HBS (20 mM HEPES, 150 mM NaCl, 3 mM EDTA, pH 7.5) was used as a running buffer and for the dilution of ligands and analytes. Biotinylated IGF-I was immobilized at a concentration of 5 nM and 10 nM in HBS at a flow rate of 5 μl/min to the strepavidin coated sensor chip resulting in signals of 40 and 110 resonance units (RU). Biotinylated IGF-II was immobilized at a concentration of 5 nM in HBS resulting in a signal of 20 RU. An empty flow cell was used as control for unspecific binding and bulk effects. The low ligand concentration was necessary to limit mass transport limitations and rebinding. For the same reason all kinetic experiments were performed at the highest possible flow rate of 100 μl/min. Each protein (wildtype and mutant IGFBPs or fragments of these proteins) was injected at four concentrations (250, 50, 10, and 2 nM). Each sample was injected for 2 min followed by dissociation in buffer flow for 4 min. After the dissociation phase the sensor chip was regenerated by injection of 10 μl 1100 mM HCl at a flow rate of 5 μl/min. The kinetic parameters were calculated using the BIAevaluation 3.0 software (BIAcore AB). After subtraction of the blank sensorgram the kinetic rate constants were calculated from a general fit of an overlay of the sensorgrams of all concentration of one analyte using the method called “1:1 binding with mass transfer”. IGF-I and IGF-II were biotinylated with a five-fold molar excess of D-biotinyl-ε-aminocaproic acid-N-hydroxysuccinimide ester using the reagents and the operation instructions of the Biotin Protein Labelling Kit (Roche Diagnostics GmbH, DE). After blocking with lysine, the reaction was dialyzed against 50 mM Na-phosphate, 50 mM NaCl, pH 7.5.

### EXAMPLE 4

### Inhibition of IGF-1-Induced IGF-IR Phosphorylation by IGFBP Mutants

Confluent monolayers of NIH3T3 cells stably expressing human IGF-IR in 3.5 cm dishes were starved in DMEM containing 0.5% dialyzed fetal calf serum. After 48 h, cells were incubated without any hormone or with 5×10⁻⁹ M IGF-I or 1×10⁻⁸ M IGF-II; each sample was preincubated with increasing concentrations of different IGF-binding proteins or fragments thereof at room temperature for 1 h. After a 10 min stimulation at 37° C., the medium was removed and cells were lysed with 250 μl of lysing buffer (20 mM Hepes, pH 7.5, 150 mM NaCl, 10% glycerol, 1% Nonidet P40, 1.5 mM MgCl₂, 1 mM EGTA (ethylene glycol-bis(2-aminoethyl)-N,N,N′,N′-tetraacetic acid, Aldrich, USA), 10 mM sodium orthovanadate, and protease inhibitor cocktail Complete (Roche Diagnostics GmbH, DE) for 10 min on ice. Subsequently, cells were scraped off the plate and the insoluble material was separated by centrifugation for 20 min at 4° C. The protein concentration of the supernatant was determined using the BCA kit from Pierce, Rockford, USA according to the manufacturer's instructions. Equal protein concentration was incubated with the SDS sample buffer (63 mM Tris-HCl, pH 6.8, 3% SDS, 10% glycerol, 0.05% bromphenolblue, 100 mM DTT), boiled for 5 min and loaded on a 7.5% SDS polyacrylamide gel. After electrophoresis the proteins were transferred on a nitrocellulose membrane which first was blocked for 1 h with the 3% BSA containing PBST (phosphate buffered saline-Tween®), then overnight incubated with 1 μg/ml monoclonal anti-phosphotyrosine antibody 3-365-10 (Roche Diagnostics GmbH, DE) in PBST that contained 3% BSA. Unbound antibody was removed by extensive washing. The blot was then incubated with 1:10000 diluted anti-mouse IgG-specific antibody conjugated with horse raddish peroxidase (Roche Diagnostics GmbH, DE). The immunoblot was developed using the ECL kit from Amersham and the concentration of IGFBP which results in 50% inhibition of the IGF-I receptor phosphorylation was determined.

### EXAMPLE 5

### Determination of the Inhibition of Tumor Cell Growth by IGFBP Mutants

MCF-7 cells (from ATCC, American type Culture Collection, Rockville, Md., U.S.A., HTB22) were used to investigate the inhibitory effect of IGFBP mutants on tumor cells. 1000 MCF-7 cells were seeded per well in medium containing 2.5% FBS (fetal bovine serum). The cells were cultured in the presence of various concentrations of IGFBPs for 48 h. The percentage of surviving cells was determined by MTT ((3-[4,5-dimethylthiazol-2-yl]-2,5-diphenyltetrazolium bromide) assay and the concentration of binding protein which results in reduction of cell survival by 50% was determined.

### EXAMPLE 6

### Mutagenesis, Expression and Purification of Mini-IGFBP-5s and Subcloning of IGFBP-4 into Pet-28a (+)

### 6.1 Buffers and Media

### Cell Growth Media:

| | |
|---|---|
| LB-medium | per 1 liter: peptone 10 g, yeast extract 5 g, NaCl 10 g, |
| | adjusted to pH 7. |
| LB-agar | per 1 liter: peptone 10 g, yeast extract 5 g, NaCl 10 g, bacto |
| | agar 15 g, adjusted to pH 7. |
| Minimal | per 1 liter: 0.5 g NaCl, 1 g citric acid monohydrate, 36 mg |
| medium | ferrous citrate (pre-dissolved in conc. HCl), 4.02 g |
| | KH₂PO₄, 7.82 g K₂HPO₄, 1 g ¹⁵N—NH₄Cl, 1.3 ml trace |
| | elements solution (per liter of the stock solution: 2.5 g |
| | H₃BO₃, 2.0 g CoCl₂, 1.13 g CuCl₂, 9.8 g MnCl₂, 2.0 g |
| | Na₂MoO₄), 1 ml Zn-EDTA solution (per ml of the stock |
| | solution: 5 mg EDTA, 8.4 mg zinc acetate), adjusted to pH |
| | 7, autoclaved. Added afterwards: 25 ml autoclaved 20% |
| | (w/v) glucose, 560 μl sterile filtered 1% (w/v) thiamine, |
| | 2 ml 1M MgSO₄. |

### Antibiotic Stocks:

| | |
|---|---|
| Ampicillin | 50 mg/ml in dist. water, 0.22 μm filtrated, |
| | stored at −20° C. |
| Kanamycin | 25 mg/ml in dist. water, 0.22 μm filtrated, |
| | stored at −20° C. |
| Chloramphenicol | 35 mg/ml in 96% ethanol, |
| | stored at −20° C. |

### Agarose-Gel Electrophoresis:

| | |
|---|---|
| TAE-buffer (50x) | 2 M Tris-HCl (pH 8.0), 2 M glacial acetic |
| | acid and 50 mM EDTA. |
| Loading buffer (3x) | 0.13% bromophenol blue, 0.13% |
| | xylene cyanol, 30% glycerol. |
| Et-Br-solution | 10 mg/ml ethidiumbromide in |
| | dd H₂O. |

### SDS-PAGE:

| | |
|---|---|
| Sample | 125 mM Tris-HCl (pH 6.8), 10% SDS, 760 mM 2- |
| buffer (5x) | mercaptoethanol, 0.13% bromophenol blue, 50% glycerol |
| | and 2 mM EDTA. |
| Staining | 0.125% CBB-R250 in 500 ml 96% ethanol and 500 ml 10% |
| solution | acetic acid. |
| Distaining | 96% ethanol, 10% acetic acid and dest. H₂O in 4:3:3 |
| solution | proportion. |

### Tricine Gels:

| | |
|---|---|
| Cathode (top) | |
| running buffer (10x) | 1 M Tris-HCl (pH 8.25), 1 M Tricine and 1% SDS. |
| Anode (bottom) | |
| running buffer (10x) | 2 M Tris-HCl (pH 8.9). |
| Separation buffer | 3 M Tris-HCl (pH 8.9) and 0.3% SDS. |
| Stacking buffer | 1 M Tris-HCl (pH 6.8) and 0.3% SDS. |
| Separation acrylamide | 48% (w/v) acrylamide, 1.5% (w/v) N,N′-methylene-bis- |
| | acrylamide. |
| Stacking acrylamide | 30% (w/v) acrylamide, 0.8% (w/v) N,N′-methylene-bis-acrylamide. |
| APS | 10% ammonium persulphate in dd H₂O. |
| Separation gel (main) | for 2 70 × 80 × 0.75 mm mini-gels: 1.675 ml H₂O, 2.5 ml |
| | separation buffer, 2.5 ml separation acrylamide, 0.8 ml |
| | glycerol, 25 μl APS and 2.5 μl TEMED. |
| Separation gel | 1.725 ml H₂O, 1.25 ml separation buffer, o.75 ml separation |
| (intermediate) | acrylamide, 12.5 μl APS and 1.25 μl TEMED. |
| Stacking gel | 2.575 ml H₂O, 0.475 ml stacking buffer, 0.625 ml stacking |
| | acrylamide, 12.5 μl 0.5 M EDTA (pH 8.0), 37.5 μl APS and |
| | 1.9 μl TEMED. |

### Protein Purification:

| | |
|---|---|
| Buffer A | 6 M guanidinium-HCl, 100 mM NaH₂PO₄, 10 mM Tris and |
| | 10 mM 2-mercaptoethanol, pH 8.0. |
| Buffer B | 6 M guanidinium-HCl, 100 mM NaH₂PO₄, 10 mM Tris and |
| | 10 mM 2-mercaptoethanol, pH 6.5 |
| Buffer C | 6 M guanidinium-HCl, 100 mM Na-acetate and 10 mM 2- |
| | mercaptoethanol, pH 4.0. |
| Buffer D | 6 M guanidinium-HCl, pH 3.0. |
| Buffer E | 200 mM arginine, 1 mM EDTA, 100 mM Tris-HCl, 2 mM |
| | reduced glutathione, 2 mM oxidised glutathione, pH 8.4. |
| PB(0) | 10 mM Na₂HPO4, 1.8 mM KH₂PO₄ and 0.05% NaN₃, pH |
| | 7.2. |
| PB(1000) | 10 mM Na₂HPO4, 1.8 mM KH₂PO₄, 0.05% NaN₃ and 1 M |
| | NaCl, pH 7.2. |
| PBS | 140 mM NaCl, 27 mM KCl, 10 mM Na₂HPO₄, 1.8 mM |
| | KH₂PO₄ and 0.05% NaN₃. |
| Thrombin | 60 mM NaCl, 60 mM KCl, 2.5 mM CaCl₂, 50 mM Tris, pH |
| cleavage | 8.0. |
| buffer | |

### 6.2 Cloning of Mini-IGFBP-5

Mini-IGFBP-5 (residues 40-92 of IGFBP-5) was subcloned from a vector containing the complete sequence of IGFBP-5 into the BamHI and PstI restriction sites of the pQE30-vector (Qiagen, Hilden, Germany). Restriction sites, a stop codon and 21 bases encoding an N-terminal thrombin cleavage site were introduced by means of PCR (Kalus, W., et al., EMBO J. 17 (1998) 6558-6572).

### 6.3 Mutagenesis of Mini-IGFBP-5

For introduction of mutations leading to substitution of Leu₆₁ by Tyr and Leu₇₄ by Met, in vitro mutagenesis was performed using QuickChange™ site-directed mutagenesis kit (Stratagene, La Jolla, Canada). Two sets of the following mutagenic oligonucleotide primers were designed for amplification of plasmid DNA and introduction of the desired point mutations:

FBP5LY:

(SEQ ID NO:1)

5′-G GGG CTG CGC TGC TAC CCC CGG CAG GAC G-3′;
RBP5LY:

(SEQ ID NO:2)

5′-C GTC CTG CCG GGG GTA GCA GCG CAG CCC C-3′;
FBP5LM:

(SEQ ID NO:3)

5′-CG CTG CAC GCC CTG ATG CAC GGC CGC GGG G-3′;
RBP5LM:

(SEQ ID NO:1)

5′-C CCC GCG GCC GTG CAT CAG GGC GTG CAG CG-3′.

The changed codons (CTC into TAC in L₆₁Y mutant and CTG into ATG in L₇₄M mutant) are presented in bold. Degenerated bases are underlined.

The reactions were set up according to the instructions found in the mutagenesis kit manual. The PCR mixtures (50 μl) contained app 50 ng of the template (pQE30 (mini-IGFBP-5), prepared by means of mini prep spin columns kit, Qiagen) and 125 ng of each of the two oligonucleotide primers. Cycling parameters for the reactions were as follows: 30 seconds at 95° C. followed by 13 cycles of 95° C. for 30 seconds, 55° C. for 1 minute and 68° C. for 7.5 min. The DpnI digestion and XL1-Blue supercompetent cells transformation was carried out strictly according to the supplier's guidelines.

Two clones of each mutant were subjected to verification by automated double stranded sequencing, which proved the existence of the expected substitutions in all 4 cases.

### 6.4 Expression of the Mutant Mini-IGFBP-5s

Electrocompetent cells BL21 were transformed with the construct carrying the mutation. From a fresh plate, a 3-ml LB culture was started and grown overday (6-7 h) in the presence of 300 μg ampicillin per ml at 37° C. From this culture 50 μl were used to inoculate 20 ml of MM. This culture was grown overnight (9-11 h). 1 l culture was inoculated in 1:50 proportion. Expression of the protein was induced at OD₆₀₀≅0.8 by addition of IPTG (1 mM final concentration). Cells were harvested after 3 h (6000×G, 20 min at 4° C.).

### 6.5 Purification of Mini-IGFBP-5

Harvested cells were resuspended in buffer A (30 ml of the buffer was used to resuspend cells from 1 l culture) and incubated at room temperature with vigorous shaking (280 RMP) for 1 h to overnight. The cells were opened by sonification (macrotip, 50% duty cycle, output control 70, 2×4 min). The cell extract was then centrifuged to pellet cell debris (65 000×G, 1 h at room temp.). The pH of the supernatant was adjusted to the value of app. 8.0. The supernatant was then mixed with pre-equilibrated with buffer A Ni-NTA Superflow matrix (Qiagen), incubated with agitation for 1 h to overnight and then loaded onto an empty column (3 ml bed volume for 1 l culture). The column was washed with buffer A followed by buffer B until a stable UV-absorption base line. Bound proteins were fractionated with 100 ml pH gradient of buffer B and C. Collected fractions were analysed by tricine gel electrophoresis (prior electrophoresis, the proteins were precipitated with 5% (w/v) TCA). Fractions containing mini-IGFBP-5 were pooled, concentrated on Amicon YM3 to 2-4 ml, and dialysed against 2 l of buffer D overnight (100 μl excess of 2-mercaptoethanol was added to the sample prior dialysis).

To promote refolding, the dialysed sample was diluted in 100 μl portions into freshly prepared, ice-cold buffer E, with vigorous stirring (in proportion 1 ml sample per 50 ml of buffer E), and left at 4° C. for 2-3 days with stirring.

The sample was concentrated on Amicon YM3 to 15-25 ml, centrifuged to get rid of a precipitated material, and dialysed overnight into 4 l of buffer PB containing 30 mM NaCl.

The solution was subsequently loaded onto pre-equilibrated with buffer PB (0) MonoS 5/5 HR cation-exchanger column (app. 1 ml) (Amersham Pharmacia, Uppsala, Sweden) at a flow rate of 1 ml/min. The column was washed with buffer PB (0). Proteins were eluted by 45 ml linear gradient of 0-70% NaCl, 1 ml fractions were collected.

The fractions containing mini-IGFBP-5 (as determined on the basis of tricine gel electrophoresis) were pooled, concentrated to 2-3 ml and loaded onto a pre-equilibrated with PBS Superdex 75 HiLoad 26/60 (app. 320 ml) gel-filtration column (Pharmacia) at a flow rate of 0.6 ml/min. Mini-IGFBP-5 was eluted as a symmetrical, single pick. Fractions containing the protein were pooled and concentrated on centricon YM3.

6.6 Subcloning into pET-28a (+)

The reason for overall low expression of the proteins from the pQE30 might be the fact that this vector is not well optimised for expression in E. coli. For instance, the vector-encoded sequences contain a cluster of 3 rare codons just downstream from the initiator codon AUG (namely, AGA, GGA and TCG, encoding Arg, Gly and Ser, respectively). The number of studies has indicated that excessive rare codon usage in a target gene may be a cause for low level expression. The impact seems to be most severe when multiple rare codons occur near the amino terminus and when they appear consecutively. Especially presence of the Arg codons AGG and AGA can have severe effects on the level of protein production. The system seems to be also not well repressed (no extra copies of a gene encoding Lac repressor), and the leaky expression may cause the observed plasmid instability. The vector carries not very efficient selective marker, Amp^{R} gene (bla), what makes possible rapid over-growing of a culture at a certain stage by cells lacking the unstable plasmid. The vector pET-28a (+) (Novagen) was then chosen as an alternative for pQE30. The plasmid is well optimised for expression of genes in E. coli, carries a strong selective marker (Kan^{R}) and is stable due to high level of repression of the target gene expression in the absence of IPTG (in a non-DE3 lysogenic strain even in the presence of the inducer).

To subclone mini-IGFBP-5 wild type, L₆₁Y and L₇₄M from pQE30 to pET-28a, the relevant fragments were excised from the vector with BamHI and HindIII (HindIII cleavage site exists in pQE30 downstream from PstI site). The excision was performed as double-digestion. Digested pET vector was 5′-dephosphorylated. Reaction mixtures were electrophorized and bands corresponding to app. 200 bp fragments excised from pQE30 (mini-IGFBP-5 wt, L₆₁Y and L₇₄M) and app 5000 bp fragment of pET-28a were cut from 1% agarose gel and purified (gel extraction kit, Qiagen). The fragments were ligated (Ligation kit, Fermentas) and XL-1 Blue Supercompetent cells were transformed with the ligation mixture.

Restriction assay carried out subsequently on isolated plasmid DNA revealed presence of fragments of expected size (restriction enzymes NcoI and PstI were used, double digestion was performed. PstI restriction site was introduced into the pET vector together with the fragment encoding mini-IGFBP-5).

Pilot-scale expression and purification experiment showed that expression of the protein of interest (mini-IGFBP-5 L₆₁Y in this case) is higher than the expression of the wild-type protein when pQE30 vector was used.

The proteins are expressed as double-fusions: they carry His-tag followed by T7-tag. It is possible to remove both tags by thrombin cleavage. Mini-IGFBP-5 after cleavage by thrombin comprises the following N-terminal amino acid sequence: GSALA (SEQ ID NO:7) (N-terminus of mini-IGFBP-5 starting from aa 40 with to additional aa from cloning with thrombin cleavage site). Vector-derived amino acids are underlined.

6.7 Subcloning of IGFBP4 from pKK177-3HB to pET-28a(+)

For subcloning of IGFBP4-2 into the NdeI and BamHI restriction sites of the pET-28a vector in-frame to a His-tag, following oligonucleotides were designed for amplification of DNA by PCR:

(SEQ ID NO:5)

IBP4NdeI: 5′-CGG AGG AAA AAC ATA TGG ATG AAG C-3′

(SEQ ID NO:6)

IBP4BamHI: 5′-GCC AAG CTT GGA TCC AGG TCG AC-3′

The restriction sites recognized by NdeI and BamHI are presented in bold. Degenerated bases are underlined.

The PCR mixture (50 μl) contained 124 ng of mixture of pKK177-3HB and Pfdx500 repressor plasmid, 130 ng of each of the primers, 1 μl dNTP mix and 2.5 U Pfu Turbo DNA polymerase (Strategene). After initial step of 30 sec. At 95° C., the reaction was cycled 30× at 95° C. for 30 seconds, 55° C. for 1 min and 68° C. for 2 min. The product of PCR was purified (PCR purification kit, Qiagen), double-digested and electrophorised. The bands corresponding to cleaved pET-28a and PCR product were excised from the gel and purified.

XL-1 Blue Supercompetent cells were transformed with the ligation mixture.

IGFBP4-2 is expressed as a N-terminal His-tag fusion protein. After thrombin cleavage, the protein comprises the following amino acid sequence: GSHMDEAIH . . . (SEQ ID NO:8). Vector derived amino acids are underlined.

The same purification routine will be used for His-tagged IGFBP-4 as for mini-IGFBP-5.

### EXAMPLE 7

### Identification of Chemical Non-Proteinaceous Compounds Binding to IGFBP-5 or IGF-I by Using the Coordinates of the Crystal Structure of the Complex of Both Molecules

FlexX version 1.9.0 was used to screen a substance library of ca. 90,000 compounds in an ACD (Available Chemicals Directory; ACD-3D 2000), choosing compounds with a molecular weight of less than 550 Daltons and containing at least one of the atoms {N, O, F, or S}. Docking searches were conducted on both molecular surfaces of the IGFBP-5 interface. Top scoring hits as judged by the FlexX standard scoring function and the proximity to binding site protein atoms were selected and tested for activity.

The top scoring compounds selected according to these these criteria for release of IGF-I from IGFBP-5 were:
Compound 1: N1-(3,4-Dichlorophenyl)-2-[2-[5-(3,5-dichlorophenyl)-2H-1,2,3,-tetraazol-2YL]A (MF: C16H11C14N7OS; MW: 491,1890 Da)
Compound 2: F-MOC-Tyr(PO3H2)-OH(C24H22NO8P; MW: 483.4110)
Compound 2A: Nα-FMOC—O-tert-butyl-L-tyrosine
Compound 2B: Nα-FMOC-L-phenylalanine
Compound 2C: Nα-FMOC—N—BOC-L-tryptophan
Compound 2D: Nα-FMOC-L-leucine
Compound 3: 4-(2,5-Dichlorophenylazo)-4′fluorosulfonyl-1-hydroxy-2-naphthanilide (MF: C23H14Cl2FN3O4S; MW: 518.3510)
Compound 4B: 5-Amino-2[(4-amino-2-carboxyphenyl)thio]benzoic acid (C14H12N2O4S; MW 304.3250)
Compound 4C: 5-Amino-2[(2-carboxyphenyl)thio]benzoic acid (C14H 11NO4S; MW 289.3100)

### EXAMPLE 8

### Release of IGF-I from the Complex with IGFBP-5 by Selected Compounds Measured by IGF-I Binding to IGF-IR Expressing Cells

Kalus, W., et al., in EMBO J. 17 (1998) 6558-6572, describe the inhibition of the binding of IGF-I to IGF-IR expressing NIH 3T3 cells by formation of an inhibitory complex. This assay was used to investigate the release of IGF-I from the inhibitory complex with IGFBP-5.

NIH 3T3 cells stably expressing human IGF-IR were grown in culture dishes in Dulbecco's modified Eagle's medium (DMEM) containing 10% fetal calf serum. Cells were washed carefully with PBS and incubated with 5 ml of 50 mM EDTA in PBS for 45 min. Cells were removed from the plate, washed once with PBS and once with binding buffer (100 mM HEPES pH 7.6, 120 mM NaCl, 5 mM KCl, 1.2 mM MgSO 4, 1 mM EDTA, 10 mM glucose, 15 mM sodium acetate, 1% dialysed BSA), and resuspended in binding buffer to determine the cell number. 5 pM ¹²⁵ I-IGF-I (Amersham) was preincubated with either 10 or 100 nM IGFBP-5 alone or in combination with 33 μM of the different compounds 1,2,3,4B and 4C at 4° C. for 1 h. Then 400 μl of the cell suspension corresponding to 2×10⁵ cells were added to give a total volume of 500 μl. After 12 h incubation at 4° C., cells were washed with binding buffer (at 4° C.). Free hormone was removed by repeated centrifugation and resuspension in the binding buffer. The 125 I radioactivity bound to the cells was determined in a gamma-counter.

As shown in FIG. 7 the labeled IGF-I binds to NIH 3T3 cells in the absence of IGFBP-5 and cell binding is inhibited by the addition of IGFBP-5. Preincubation of the complex of IGFBP-5 and IGF-I with the selected compounds results in release of IGF-I from the complex by compound 3 and consequently binding of IGF-I to the IGF-IR expressing cells.

### EXAMPLE 9

### Release of IGF-I from the Complex with IGFBP-5 by Selected Compounds Measured by IGF-IR Activation

Kalus, W., et al., in EMBO J. 17 (1998) 6558-6572, describe the inhibition of the activation and autophosphorylation of the IGF-IR by IGF-I in the presence of IGFBP-5. This assay was used to further investigate the release of IGF-I from the inhibitory complex with IGFBP-5 by compound 3. Binding of compound 3 to IGFBP-5 and dissociation of the complex of the binding protein with IGF-I should result in an activation and autophosphorylation of the IGF-IR in the presence of IGFBP-5.

Confluent monolayers of the NIH 3T3 cells stably expressing human IGF-IR in 3.5 cm dishes were starved in DMEM containing 0.5% dialysed fetal calf serum. After 48 h, cells were incubated without any hormone or with 10 nM IGF-I. Samples were preincubated with 100 nM IGFBP-5 and increasing concentrations of compound 3 from 0 to 50 μM at room temperature for 1 h. After a 10 min stimulation at 37° C., the medium was removed and cells were lysed with 250 μl of lysing buffer (20 mM HEPES pH 7.5, 150 mM NaCl, 10% glycerol, 1% NP-40, 1.5 mM MgCl 2, 1 mM EGTA), 10 mM sodium orthovanadate, and protease inhibitor cocktail Complete (Roche Molecular Biochemicals) for 10 min on ice. Subsequently, cells were scraped off the plate and the insoluble material was separated by centrifugation for 20 min at 4° C. The protein concentration of the supernatant was determined using the BCA kit from Pierce according to the manufacturer's instructions. Equal protein concentration was incubated with the SDS sample buffer (63 mM Tris-HCl pH 6.8, 3% SDS, 10% glycerol, 0.05% bromophenolblue, 100 mM DTT), boiled for 5 min and loaded on a 7.5% SDS-polyacrylamide gel. After electrophoresis the proteins were transferred on a nitrocellulose membrane which first was blocked for 1 h with the 3% BSA containing phosphate-buffered saline-Tween (PBST), then incubated overnight with 1 mg/ml monoclonal anti-phosphotyrosine antibody 4G10 (Upstate Biotechnology), polyclonal anti-phospho-AKT antibody (New England Biolabs) or polyclonal anti-IGF-IR(C-20, Santa Cruz Biotechnology) in PBST that contained 3% BSA. Unbound antibody was removed by extensive washing. The blot was then incubated with 1:10 000 diluted anti-mouse IgG-specific antibody or 1:5000 diluted anti-rabbit specific antibody conjugated with horse radish peroxidase (both Roche Molecular Biochemicals). The immunoblot was developed using the ECL kit from Amersham.

As shown in FIG. 8 the autophosphorylation of IGF-IR by IGF-I is inhibited in the presence of IGFBP-5. The addition of compound 3 to the inactive complex of IGFBP-5 and IGF-I results in an increased autophosphorylation of the receptor at 50 uM compound 3.

### EXAMPLE 10

### Detection of Ligand Binding

Ligand binding was detected by acquiring ¹⁵N—HSQC spectra. All NMR spectra were acquired at 300 K on Bruker DRX600 spectrometer. The samples for NMR spectroscopy were concentrated and dialyzed against PBS buffer. Typically, the sample concentration was varied from 0.3 to 1.0 mM. Before measuring, the sample was centrifuged in order to sediment aggregates and other macroscopic particles. 450 μl of the protein solution were mixed with 50 μl of D₂O (5-10%) and transferred to an NMR sample tube. The stock solutions of compounds were 100 mM either in water or in perdeuterated DMSO. pH was maintained constant during the whole titration. The binding was monitored by observation of the changes in the ¹⁵N—HSQC spectrum. Dissociation constants were obtained by monitoring the chemical shift changes of the backbone amide of several amino acid residues (Table 9) as a function of ligand concentration. Data were fit using a single binding site model. In the same way dissociation constants for derivatives of compound 2 are estimated (Table 10).

TABLE 9
Dissociation constant calculations for compound 2 or
DMSO binding to IGFBP-5
using data from distinct amino acid residues

| | ligand in DMSO K_{D} | ligand in PBS K_{D} | |
|---|---|---|---|
| residue | [mM] | [mM] | DMSO K_{D} [mM] |
| Y50 | 1.58 ± 0.09 | 1.82 ± 0.95 | 648 ± 370 |
| L73 | 1.31 ± 0.17 | 2.93 ± 1.41 | 541 ± 306 |
| S85 | 1.38 ± 0.10 | 2.33 ± 0.94 | 650 ± 373 |
| Y86 | 1.90 ± 0.17 | 1.72 ± 0.99 | 783 ± 498 |
| R87 | 1.64 ± 0.12 | 2.36 ± 1.00 | 921 ± 662 |
| K91 | 2.42 ± 0.18 | 2.12 ± 1.03 | 719 ± 434 |
| average: | 1.71 ± 0.37 | 2.21 ± 0.40 | 710 ± 120 |

TABLE 10
Dissociation constants calculated for compound 2
and its derivatives binding to
IGFBP-5 using changes in chemical shift for the residue L81

| compound | chemical name | K_{D} [mM] |
|---|---|---|
| 2 | Nα-FMOC-O-phospho-L-tyrosine | 2.78 ± 0.30 |
| 2A | Nα-FMOC-O-tert-butyl-L-tyrosine | 0.718 ± 0.079 |
| 2B | Nα-FMOC-L-phenylalanine | 1.075 ± 0.507 |
| 2C | Nα-FMOC-N-BOC-L-tryptophan | 0.0432 ± 0.0115 |
| 2D | Nα-FMOC-L-leucine | 1.088 ± 0.519 |

### LIST OF REFERENCES

Abrahams, J. P., and Leslie, A. G. W., Acta. Cryst. D52 (1996) 30-42
Adams, M. J., et al., Nature 224 (1969) 491-492
Bach, L. A., et al., J. Biol. Chem. 268 (1993) 9246-9254
Bacon et al., J. Mol. Biol. 225 (1992) 849-858
Baxter, R. C., et al., J. Biol. Chem. 267 (1992) 60-65
Blundell, T. L., et al., Nature 326 (1987) 347
Blundell, T. L., Proc. Natl. Acad. Sci. USA 75 (1978) 180-184
Bohm, H. J., et al., J. Comput. Aided Mol. Des. 6 (1992) 61-78 and 593-606
Brünger, A. T., et al., Acta Crystallogr. D54 (1998) 905-921
Butt, A. J., et al., J. Biol. Chem. 275 (2000) 39174-39181
Byun, D., et al., J. Endocrinology 169 (2001) 135-143
Chernausek, S. D., et al., J. Biol. Chem. 270 (1995) 11377-11382
Conover, C. A., et al., in J. Biol. Chem. 270 (1995) 4395-4400
Cooke, R. M., et al., Biochemistry 30 (1991) 5484-5491
de la Fortelle, E., and de Bricogne, G., Methods Enzymol. 276 (1997) 472-494
Fanayan, S., et al., J. Biol. Chem. 275 (2000) 39146-39151
Gill, R., et al., Prot. Eng. 9 (1996) 1011-1019
Goodford, P. J., J. Med. Chem. 27 (1984) 557
Goodford, P. J., J. Med. Chem. 28 (1985) 849-857
Hankinson, S. E., et al., Lancet 351 (1998) 1393-1396
Holly, J., Lancet 351 (1998) 1373-1374
Hwa, V., et al., The IGF binding protein superfamily, In: Rosenfeld, R. G., and Roberts, C. T. (eds.), The IGF system, Molecular Biology, Physiology, and Clinical Applications (1999), Humana Press, Totowa, pp. 315-327
Imai, Y., et al., J. Biol. Chem. 275 (2000) 18188-18194
Jansson, M., et al., Biochemistry 36 (1997) 4108-4117
Jones, J. L., and Clemmons, D. R., Endocr. Rev. 12 (1995) 10-21
Kalus, W., et al., EMBO J. 17 (1998) 6558-6572
Khandwala, H. M., et al., Endocr. Rev. 21 (2000) 215-244
Kramer, B., et al., Proteins: Structure, Functions and Genetics 37 (1999) 228-241
Kuntz, I. D., et al., J. Mol. Biol. 161 (1982) 269-288
Laajoki, L. G., et al., J. Biol. Chem. 275 (2000) 10009-10015
Leslie, A. G. W., Molecular Data in Processing, in: Moras, D., Podjarny, A. D., and Thierry, J. C. (eds.), Crystallographic Computing 5 (1991), Oxford University Press, Oxford, UK, pp. 50-61
Loddick, S. A., et al., Proc. Natl. Acad. Sci. USA 95 (1998) 1894-1898
Lowman, H. B., et al., Biochemistry 37 (1998) 8870-8878
Luethi, C., et al., Eur. J. Biochem. 205 (1992) 483-490
Martin, J. L., and Baxter, R. C., IGF binding proteins as modulators of IGF actions; in: Rosenfeld, R. G., and Roberts, C. T. (eds.), The IGF system, Molecular Biology, Physiology, and Clinical Applications (1999), Humana Press, Totowa, pp. 227-255
Mazerbourg, S., et al., Endocrinology 140 (1999) 4175-4184
Murray-Rust, J., et al., BioEssays 14 (1992) 325-331
Remington's Pharmaceutical Sciences, 18^{th} ed., 1990, Mack Publishing Company, edited by Oslo et al. (e.g. pp. 1435-1712)
Sato, A., et al., Int. J. Pept. Protein Res. 41 (1993) 433-440
Sheldrick, G., Tutorial on automated Patterson interpretation to find heavy atoms, in: Moras, D., Podjarny, A. D., and Thierry, J. C. (eds.), Crystallographic Computing 5 (1991), Oxford University Press, Oxford, UK, pp. 145-157
Sheridan, R. P., and Venktaraghavan, R., Acc. Chem. Res. 20 (1987) 322
SwissProt Database (http://www.expasy.ch)
Terasawa, H., et al., EMBO J. 13 (1994) 5590-5597
Torres, A. M., et al., J. Mol. Biol. 248 (1995) 385-401
van den Berg, C. L., et al., Eur. J. Cancer 33 (1997) 1108-1113
Verlinde, C., and Hol, W., Structure 2 (1994) 577
Wetterau, L. A., et al., Mol. Gen. Metab. 68 (1999) 161-181
Wolk, A., Lancet 356 (2000) 1902-1903

1. A crystal suitable for X-ray diffraction, comprising a complex of insulin-like growth factor I or II and a polypeptide consisting of amino acids 40-92 of SEQ ID NO:15, or a fragment consisting of at least the amino acids from the 9^{th} to 12^{th} cysteines of SEQ ID NO:15 wherein the complex exhibits restricted conformation mobility.

2. A crystal of claim 1, which effectively diffracts X-ray for the determination of the atomic coordinates of the complex to a resolution of 1.5 to 3.5 Å.

3. A method for producing a crystal suitable for X-ray diffraction, comprising
(a) contacting insulin-like growth factor I or insulin-like growth factor II with a polypeptide consisting of amino acids 40-92 of SEQ ID NO:15, or a fragment consisting of at least the amino acids from the 9^{th} to 12^{th} cysteines of SEQ ID NO:15 wherein the complex exhibits restricted conformation mobility, and
(b) obtaining a crystal from the complex so formed suitable for X-ray diffraction.

4. A method for the determination of the atomic coordinates of a crystal suitable for X-ray diffraction comprising
(a) contacting insulin-like growth factor I or insulin-like growth factor II with a polypeptide consisting of amino acids 40-92 of SEQ ID NO:15, or a fragment consisting of at least the amino acids from the 9^{th} to 12^{th} cysteines of SEQ ID NO:15 wherein the complex exhibits restricted conformation mobility;
(b) obtaining a crystal from the complex so formed suitable for X-ray diffraction; and
(c) determining the atomic coordinates of said crystal.

5. A method for identifying a mutant of an insulin-like growth factor binding protein having an enhanced binding affinity for insulin-like growth factor I or insulin-like growth factor II, comprising
(a) constructing a three-dimensional structure of the complex of insulin-like growth factor I or insulin-like growth factor II and a polypeptide consisting of amino acids 40-92 of SEQ ID NO:15, or a fragment consisting of at least the 9^{th} to 12^{th} cysteines of SEQ ID NO:15, based on the atomic coordinates of a crystal consisting of insulin-like growth factor I and said polypeptide or fragment;
(b) employing said three-dimensional structure and modeling methods to identify said mutant of an insulin-like growth factor binding protein in which a residue within a distance of 5 Å to a hydrophobic amino acid residue of insulin-like growth factor I or insulin-like growth factor II is modified in that the hydrophobic interaction between insulin-like growth factor I or insulin-like growth factor II and said mutant of an insulin-like growth factor binding protein is enhanced;
(c) producing said mutant; and
(d) assaying said mutant to determine said enhanced binding affinity for insulin-like growth factor I or insulin-like growth factor II.

6. A mutant of an insulin-like growth factor binding protein having the sequence set forth in SEQ ID NO:15 wherein the mutant comprises one or more of the mutations as depicted in Table 5.

7. A mutant of an insulin-like growth factor binding protein having the sequence set forth in SEQ ID NO:15 wherein the mutant comprises one or more mutations of amino acid residues 49, 70 and/or 73 according to Table 5.

8. A method for identifying a non-proteinaceous compound capable of binding to an insulin-like growth factor binding protein, comprising
(a) constructing a three-dimensional structure of a complex of insulin-like growth factor I or insulin-like growth factor II and a polypeptide consisting of amino acids 40-92 of SEQ ID NO:15, or a fragment consisting of at least the amino acids from the 9^{th} to 12^{th} cysteines of SEQ ID NO:15, based on the atomic coordinates of a crystal consisting of insulin-like growth factor I and said polypeptide or fragment;
(b) employing said three-dimensional structure and modeling methods to identify a non-proteinaceous compound forming a complex with said polypeptide or fragment by hydrophobic binding with amino acids 49, 50, 70, 71 and 74 of SEQ ID NO:15;
(c) producing said compound;
(d) determining the binding between the compound and said polypeptide or fragment.

9. A crystal of claim 1, wherein the crystal is arranged in the cubic space group P2₁3 having unit cell dimensions of 74.385 Å.×74.385 Å×74.385 Å.

10. A crystal of claim 2, wherein the crystal is arranged in the cubic space group P2₁3 having unit cell dimensions of 74.385 Å.×74.385 Å×74.385 Å.


SEQUENCE LISTING (16 sequences)

SEQ ID NO: 1 - DNA, 29 bp, Artificial Sequence
  Description of Artificial Sequence Synthetic primer
ggggctgcgctgctacccccggcaggacg

SEQ ID NO: 2 - DNA, 29 bp, Artificial Sequence
  Description of Artificial Sequence Synthetic primer
cgtcctgccgggggtagcagcgcagcccc

SEQ ID NO: 3 - DNA, 30 bp, Artificial Sequence
  Description of Artificial Sequence Synthetic primer
cgctgcacgccctgatgcacggccgcgggg

SEQ ID NO: 4 - DNA, 30 bp, Artificial Sequence
  Description of Artificial Sequence Synthetic primer
ccccgcggccgtgcatcagggcgtgcagcg

SEQ ID NO: 5 - DNA, 25 bp, Artificial Sequence
  Description of Artificial Sequence Synthetic primer
cggaggaaaaacatatggatgaagc

SEQ ID NO: 6 - DNA, 23 bp, Artificial Sequence
  Description of Artificial Sequence Synthetic primer
gccaagcttggatccaggtcgac

SEQ ID NO: 7 - protein, 5 aa, Artificial Sequence
  Description of Artificial Sequence Synthetic peptide
GSALA

SEQ ID NO: 8 - protein, 9 aa, Artificial Sequence
  Description of Artificial Sequence Synthetic peptide
GSHMDEAIH

SEQ ID NO: 9 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 10 - protein, 66 aa, Homo sapiens
AYRPSETCGGELVDTLQFVCGDRGFYFSRPASRVSRRSRGIVEECCFRSCDLALLETYCATPAKSE

SEQ ID NO: 11 - protein, 112 aa, Homo sapiens
QVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEIT

SEQ ID NO: 12 - protein, 132 aa, Homo sapiens
GGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENH

SEQ ID NO: 13 - protein, 123 aa, Homo sapiens
AGASSGGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSV

SEQ ID NO: 14 - protein, 117 aa, Homo sapiens
LLAAGPGPSLGDEAIHCPPCSEEKLARCRPPVGCEELVREPGCGCCATCALGLGMPCGVYTPRCGSGLRCYPPRGVEKPLHTLMHGQGVCMELAEIEAIQESLQPSDKDEGDHPNNS

SEQ ID NO: 15 - protein, 115 aa, Homo sapiens
YAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIERDSREHEEPTTSEMA

SEQ ID NO: 16 - protein, 110 aa, Homo sapiens
LAASPGGALARCPGCGQGVQAGCPGGCVEEEDGGSPAEGCAEAEGCLRREGQECGVYTPNCAPGLQCHPPKDDEAPLRALLLGRGRCLPARAPAVAEENPKESKPQAGTA